SEQUENCE LISTING (16 sequences)

SEQ ID NO: 1 - protein, 192 aa, Hepatitis C virus Type 1a
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDA

SEQ ID NO: 2 - protein, 363 aa, Hepatitis C virus Type 1a
ETHVTGGSAGHTTAGLVGLLTPGAKQNIQLINTNGSWHINSTALNCNDSLTTGWLAGLFYRHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 3 - protein, 214 aa, Hepatitis C virus Type 1b
MGCSFSIFLLALLSCLTIPASAYEVRNVSGVYHVTNDCSNASIVYEAADMIMHTPGCVPCVRENNFSRCWVALTPTLAARNTSVPTTTIRRHVDLLVGAAAFCSAMYVGDLCGSVFLVSQLFTFSPRRHETVQDCNCSIFPGPVTGHRMAWDMMMNWSPTTAIVVSQLLRIPQAVVDMVAGAHWGVLAGLAYYSMVGNWAKVLIVMLLFAGVDG

SEQ ID NO: 4 - protein, 363 aa, Hepatitis C virus Type 1b
ETHVTGGKQSRDTHGFVSLFTLGPSQKIQLVNSNGSWHINRTALNCNDSLNTGFLAALFYTHRFNSSGCPERMSSCRRIDAFAQGSGPITYTEPDSPDQRPYCWHYAPRPCGIVPASKVCGPVYCFTPSPVVVGTTDRSGVPTYSWGENETDVLLLNNTRPPQGNWFGCTWMNSTGFTKTCGGPPCKIGGVGNNTLTCPTDCFRKHPEATYTKCGSGPWLTPRCMVDYPYRLWHYPCTVNFTIFKVRMYLGGVEHRLNAACNWTRGERCHLEDRDRSELSPLLLSTTEWQILPCSFTTLPALSTGLIHLHQNIVDVQYLYGIGSAVVSFVIKWEYVVLLFLLLADARVCACLWMMLLIAQAEA

SEQ ID NO: 5 - protein, 189 aa, Hepatitis C virus Type 2a
VRNSSGLYHVTNHCPNSSIVYETADTILHSPGCVPCVREGNASRCWVAVAPTVATRDGKLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVD

SEQ ID NO: 6 - protein, 344 aa, Hepatitis C virus Type 2a
YVTGGAAAKGASTFAGLFAPGSRQNIQLINTNGSWHINRTALNCNASLETGWLVGLFYYNKFNSSGCTERMASCRPLADFDQGWGPITYANGSGPEHRPYCWHYPPKPCGIVPAQNVCGPVYCFTPSPVVVGTTDRSGAPTYNWGSNDTDVFVLNNTRPPLGNWFGCTWMNSSGFTKVCGAPPCNIGGVGNNTLHCPTDCFRKHPEATYSRCGSGPWITPRCLVDYPYRLWHYPCTLNYTLFKVRMYVGGVEHRLGVACNWTRGERCDLDDRDRSELSPLLLSTTQWQVLPCSFTTLPALTTGLIHLHQNIVDVQYLYGVGSSIVSWAIKWEYVILLFLLLADA

SEQ ID NO: 7 - protein, 214 aa, Hepatitis C virus Type 2b
MGCSFSIFLLALLSCLTVPASAVEVRNVSSSYYATNDCSNSSITWQLNNAVLHLPGCVPCENDNGTLHCWIQVTPNVAVKHRGALTHNLRTHVDMIVMAATVCSALYVGDMCGAVMIVSQALIISPERHNFTQECNCSIYQGHITGHRMAWDMMLNWSTTLTMILAYAARVPELVLEVVFGGHWGVVFGLAYFSMQGAWAKVIAILLLVAGVDA

SEQ ID NO: 8 - protein, 367 aa, Hepatitis C virus Type 2b
ETHTTGSAAGHAARGFANLLSPGSKQKISLINTNGSWHINRTALNCNDSLQTGFLASLFYVNNFNSSGCPERMSSCRGLDDFRIGWGTLEYETNVTNEEDMRPYCWHYPPKPCGIISARTVCGPVYCFTPSPVVVGTTDEQGLPTYSWGENETDVFLLNSTRPPQGAWFGCTWMNGTGFTKTCGAPPCRIRRDFNSTLDLLCPTDCFRKHPDTTYLKCGAGPWLTPRCLVDYPYRLWHYPCTVNFTIFKVRMYVGGVEHRLSAACNFTRGDRCRLEDRDRGQQSPLLHSTTEWAVLPCSFSDLPALSTGLLHLHQNIVDVQYLYGLTPAITKYIVKWEWVVLLFLLLADARVCACLWMLIILGQAEA

SEQ ID NO: 9 - protein, 214 aa, Hepatitis C virus Type 3a
MGCSFSIFLLALFSCLIHPAASLEWRNTSGLYILTNDCPNSSIVYEADDVILHTPGCIPCVQDGNTSTCWTSVTPTVAVRYVGATTASIRSHVDLLVGAATMCSALYVGDACGAVFLVGQAFTFRPRRHQTVQTCNCSLYPGHLTGHRMAWDMMMNWSPAVGMVVAHVLRLPQTLFDIIAGAHWGILAGLAYYSMQGNWAKVVIIMVMFSGVDA

SEQ ID NO: 10 - protein, 369 aa, Hepatitis C virus Type 3a
STYTTGGQAARGARGIAGLFDLGARQNLQLVNTNGSWHINRTALNCNESINTGFIAGLFYYHKFNSTGCPQRLSSCKPITSFRQGWGPLTDANITGSSDDKPYCWHYAPRPCESVPASKVCGPVYCFTPSPVVVGTTDAKGVPTYTWGANETDVFLLNSLRPPKGRWFGCTWMNSTGFTKTCGAPPCNIYGGGGNSKNESDLFCPTDCFRKHPEATYSRCGAGPWLTPRCMVDYPYRLWHYPCTVNFTLFQVRMFVGGFEHRFTAACNWTRGERCDIEDRDRSEQHPLLHSTTELAILPCSFTPMPAWSTGLIHLHQNIVDVQYLYGVGSGMVGWALKWEFVVLVFLLLADARVCVALWLMLMISQAEA

SEQ ID NO: 11 - protein, 214 aa, Hepatitis C virus Type 4
MGCSFSIFLLALLSCLTVPASAINYRNVSGIYHVTNDCPNSSIVYETEHHILHIPGCVPCVRVGNQSSCWVALTPTVATPYIGAPLESLRSHVDLMVGAATACSALYIGDLCGGVFLVGQMFSFRPRRHWTTQDCNCSIYTGHITGHRMAWDMMMNWSPTATLVLAQLMRIPGALVDLLAGGHWGVLVGVAYFCMQANWAKVILVLFLFAGVDA

SEQ ID NO: 12 - protein, 363 aa, Hepatitis C virus Type 4
QTRLTGGTPAHGAQSIAKLFSAGPRQNVQLINSNGSWHINRTALNCNDSLNTGFLTGLFYVSRFNSSGCPERLASCKSLDSFNQGWGPLGAANISGPSDDKPYCWHYAPRPCGIVPASSVCGPVYCFTPSPVVVGTTDRFGVPTYTWGDNESDVFLLNATRPPRGAWFGCVWMNSTGFTKTCGAPPCEVKTNNGTWSCPTDCFRKHPATTYTKCGSGPWITPRCLVHYPYRLWHFPCTLNFTIFKVRTYIGGIEHRMEVACNWTRGEVCGLEHRDRAELSPLLLSTTTWQVLPCSFTTLPALSTGLIHLHQNIVDVQYLYGVGSAVVSWALKWEYVVLVFLLLADARISACLWMMFMVSQVEA

SEQ ID NO: 13 - protein, 214 aa, Hepatitis C virus Type 5
MGCSFSIFILALLSCLTVPASAVPYRNASGIYHVTNDCPNSSIVYEADNLILHAPGCVPCVREGNVSRCWVQITPTLSAPNLGAVTAPLRRAVDYLAGGAALCSALYVGDACGAVFLVGQMFTYRPRQHTTVQDCNCSIYSGHVSGHRMAWDMMMNWSPTSALLVAQMLRIPQVVMDIIAGGHWGVLFAAAYFASAANWAKVVLVLFLFAGVDG

SEQ ID NO: 14 - protein, 364 aa, Hepatitis C virus Type 5
NTQTVGGSAAYSTSGLASLFSPGPKQNLQLIQNGSSWHINRTALNCNDSLKTGFIAGLMYVYKFNSTGCPERMASCRPLTAFDQGWGTISYANISGPSDDKPYCWHYPPRPCGVVPAREVCGPVYCFTPSPVVVGTTDRKGYPTYNWGENDTDVLLLNNTRPPTGNWFGCTWMNASGFVKTCGAPPCILGPTGNNSLKCPTDCFRKHPDATYTRCGSGPWLTPRCLVDYPYRLWHYPCTVNYTIFKVRMFIGGLEHRFDAACNWTRGERCDLDDRDRAELSPLLHTTTQWAILPCSFTPTPALSTGLIHLHQNIVDTQYLYGLSSSIVSWAVKWEYIVLAFLLLADARVCTCLWIMLLVCQAEA

SEQ ID NO: 15 - protein, 214 aa, Hepatitis C virus Type 6
MGCSFSIFLLALLSCLTTPASALTYGNSSGLYHLTNDCPNSSIVLEADAMILHLPGCLPCVRVDNRSTCWHAVSPTLAIPNASTPATGFRRHVDLLAGAAVVCSSLYIGDLCGSLFLAGQLFTFQPRRHWTVQDCNCSIYTGHVTGHRMAWDMMMNWSPTTTLVLSSILRVPEICASVILGGHWGILLAVAYFGMAGNWLKVLAVLFLFAGVEA

SEQ ID NO: 16 - protein, 368 aa, Hepatitis C virus Type 6
TTTTAHEAARATAGFVGLFTSGARQKLQLINTNGSWHINRTALNCNDSLQTGFIASLFYFNKFNSSGCPERMAACKPLADFRQGWGQITYKVNISGPSDDRPYCWHYAPRSCDVVPARTVCGPVYCFTPSPVVVGTTDRRGNPTYTWGENETDVFMLESLRPPTGGWFGCTWMNSTGFTKTCGAPPCQIVPGDYNSSANELLCPTDCFRKHPEATYQRCGSGPWLTPRCLVDYPYRLWHYPCTVNFTVHKVRMFVGGIEHRFDAACNWTRGERCELHDRDRIEMSPLLFSTTQLAILPCSFSTMPALSTGLIHLHQNIVDVQYLYGVSSSVTSWVVKWEYIVLMFLVLADARICTCLWLMLLITNVEA

(19) United States
(12) Patent Application Publication
(10) Pub. No.: US 2014/0088184 A1
(43) Pub. Date: Mar. 27, 2014

(54) Use of Epigallocatechin Gallate as an Antiviral Agent against infections by the Hepatitis C Virus

(71) Applicants: Karin Seron, Saint-Omer (FR); Jean Dubuisson, Faches-Thumesnil (FR); Yves Rouille, Wasquehal (FR); Czeslaw Wychowski, Meurchin (FR)

(72) Inventors: Karin Seron, Saint-Omer (FR); Jean Dubuisson, Faches-Thumesnil (FR); Yves Rouille, Wasquehal (FR); Czeslaw Wychowski, Meurchin (FR)

(73) Assignees: INSTITUT PASTEUR DE LILLE, Lille Cedex (FR); CENTRE NATIONAL DE LA RECHERCHE SCIENTIFIQUE (C.N.R.S.), Paris (FR)

(21) Appl. No.: 14/008,031

(22) PCT Filed: Mar. 28, 2012
(86) PCT No.: PCT/EP12/55533

§ 371 Date: Dec. 11, 2013

(30) Foreign Application Priority Data
Mar. 28, 2011 (FR) 1152536

(51) Int. Cl. A61K 31/353; A61K 45/06

(52) U.S. Cl. CPC A61K 31/353; A61K 45/06
USPC 514/456; 435/238

(57) ABSTRACT

The present invention relates to a flavonoid compound having the formula I, where R3, R5 and/or R7 is a group having the formula II, or R1 and R2 are both OH groups, or to one of the pharmaceutically acceptable salts or esters thereof, for use as an antiviral agent in the treatment and/or prevention of a hepatitis C virus (HCV) infection. The invention also relates to an ex vivo method for reducing the infectivity of HCV or for inactivating HCV, including a step of contacting said hepatitis C virus with a compound having the formula (I).

### CROSS-REFERENCE TO RELATED APPLICATIONS

This application is a National Stage application of International Patent Application No. PCT/EP2012/055533, filed on Mar. 28, 2012, which is incorporated by reference herein in its entirety, which claims the benefit of French Application No. 1152536 filed Mar. 28, 2011, which is incorporated by reference herein in its entirety.

### INCORPORATION OF SEQUENCE LISTING

A text file of the Sequence Listing contained in the file named “13P2436_Seq List_ST25.txt” which is 1,527 bytes (measured in MS-Window®) in size and which was created on Sep. 24, 2013, is electronically filed herewith and is incorporated by reference in its entirety. This sequence listing consists of SEQ ID NO: 1-16.

### BACKGROUND OF THE INVENTION

The present invention relates to a flavonoid compound or to one of the pharmaceutically acceptable salts or esters thereof, for use as an antiviral agent in the treatment and/or prevention of a hepatitis C virus (HCV) infection. The invention also relates to an ex vivo method for inactivating HCV, including a step of contacting said hepatitis C virus with a flavonoid compound or a pharmaceutically acceptable salt or ester thereof.

The hepatitis C virus is a major cause of chronic liver disease that affects about 3% of the world population. Infected patients have a high risk of developing cirrhosis or hepatocellular carcinoma requiring the use of liver transplant.

The hepatitis C virus is a virus with linear single stranded RNA and positive polarity belonging to the Flaviviridae family in the genus Hepacivirus. This virus is the only known member of the genus Hepacivirus.

There are six major HCV genotypes and more than 50 subtypes that are differentially distributed geographically. By way of an indication, the HCV genotype 1 is predominant in Europe and the United States, with genotype 4 predominant in the Middle East and Africa. The high genetic heterogeneity of HCV linked to the strong propensity of the virus to mutate has important clinical and diagnostic implications and may explain the difficulties in the development of vaccines and the lack of response to current treatments.

In fact, the treatments of reference, namely, a combination of Pegylated interferon (IFN) alpha (peginterferon alfa) and an antiviral such as ribavirin, are non specific and moderately effective. Thus, remission is observed in 40% to 80% of patients infected based on the concerned viral genotype, genotype 1 being the most resistant to available therapies. In addition to its limited efficacy, this combination therapy has significant side effects. The most frequently observed adverse effects of IFN therapy include flu like symptoms such as fever, muscle or joint pain or weight loss. In addition, neuropsychiatric side effects, including mood swings and even suicidal ideation in psychosis are described. Pegylated interferon can also induce autoimmune diseases or may even aggravate preexisting autoimmune disorders. A common side effect frequently observed with ribavirin is anemia, in particular haemolytic anemia, which requires continuous monitoring of blood parameters during the treatment.

These various adverse side effects are a major reason for rejection of the treatment by patients. There is therefore a need for treatment options that are more effective, practical and better tolerated.

A large majority of anti-HCV antiviral therapies developed in the prior art are dependent on the viral genotype or even on the viral subtype. Such is the case of viral protease or polymerase specific inhibitors of viral metabolism. There is therefore also a need for inhibitors independent of the viral genotype of HCV.

In order to resolve these problems of the prior art, the inventors have shown that flavonoids containing a 3,4,5-trihydroxy-phenyl group with the formula

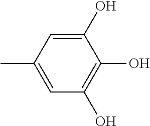

and more particularly certain catechins had a specific HCV antiviral effect regardless of the viral genotype thereof.

Flavonoid compounds are naturally occurring substances in plants and especially in most fruits and vegetables. They are responsible for the various colours of flowers and fruits and represent an important source of dietary antioxidants in food. They constitute a subclass of polyphenols. Many flavonoid compounds have therapeutic properties and are well known for their excellent potential with respect to inhibiting the processes of angiogenesis and formation of metastases. Amongst the flavonoids, and more specifically amongst the 3-hydroxy flavonoids, the flavanols, anthocyanins, leucoanthocyanins and catechins are distinguished as subclasses. Catechins are considered to be the most studied flavonoid compounds.

Catechins, their derivatives, or their degradation products, possess multiple therapeutic properties. Their therapeutic effects have been widely described whether it be in the form of an isolated catechin or in the form of extracts of green tea from which they are mostly derived. Purely on an indicative basis, catechin. (−)-epicatechin (EC), (−)-epigallocatechin (EGC), (−)-epicatechin-3-gallate (ECG) and epigallocatechin-3-gallate (EGCG) may be cited. As such, catechins are thus described as having a preventative effect on obesity or cardiovascular diseases such as atherosclerosis. They are also described as having an antibacterial antioxidant, anticarcinogenic, or antiviral effect (Khan N et al, Life Sci. 2007 81, 19-33).

The modes of action of these catechins are diverse. Thus, their anti-carcinogenicity in the case of breast cancer or prostate cancer, is linked to the cytotoxic effect of EGCG on cancer cells (Stuart E C et al, Life Sci. 2006. 79: 2329-36). The antiviral effect of catechins are believed to pass through various metabolic pathways. In the case of the influenza or hepatitis B virus, EGCG and/or ECG are believed to inhibit viral replication and/or transcription (Song J M et al, Antiviral Research 2005 68 66-74; Xu et al, 2007). With regard to the influenza virus or Human Immunodeficiency Virus Type 1 (HIV-1), EGCG inhibits binding to their respective host cells (Song J M et al, Antiviral Research 2005 68 66-74; and Nance C L et al, J Allergy Clin Immunol 2009 123:459-65). In the case of the Herpes Simplex Virus (types 1 and 2), EGCG induces viral death by creating pores in response to binding to envelope glycoproteins (Isaacs C E et al, Antimicrob Agents Chemother. 2008 52:962-70).

With respect to the liver, catechins are described as hepato-protective in particular because of their antioxidant property. In addition, they are often associated with the treatment therapies for liver diseases. Hence catechins are described in the treatment of viral hepatitis type B or type C as reducing cell necrosis (Patrick L et al, Altern Med. Rev. 1999 August; 4(4): 220-38). This effect is independent of the viral infection itself, but enables reduction of the necrotic inflammation of the liver associated with it.

Catechins have been proposed for the treatment of diseases involving the route of immunosuppression mediated by indoleamine 2,3-dioxygenase (IDO), among which are certain cancers as also viral infections including hepatitis C (WO 2008115804). This course of treatment is thus non specific and indirect as it entails treatment by immunomodulation.

The prior art also describes treatment of hepatitis C by use of an antiviral agent in combination with an inhibitor of cellular proteasome. Among the cellular proteasome inhibitors mentioned, EGCG is also featured. The mechanism of action and the advantage of a proteasome inhibitor in this treatment nevertheless remains to be described (WO2011/009961).

Catechins are also mentioned in the treatment of hepatitis C in a polymeric form (US 2010/0055065). This polyphenol polymer is described as exhibiting antiviral activity by inhibiting the replication of the HCV virus, subject to the proviso of containing at least 3 monomers. A number of polyphenols and more particularly all of the catechins are considered as potential monomers. However, during the course of this study, only the genotype 1b replicon as described by Lohmann et al Science, 1999, has been used by the authors.

However, none of documents of the prior art describes a molecule with an HCV specific antiviral effect that is effective, while at the same time having few or no adverse side effects on the patient treated and which is specific to the hepatitis C virus regardless of the viral genotype concerned.

In order to respond to all of the problems of the prior art, the invention relates to a compound having the formula (I)

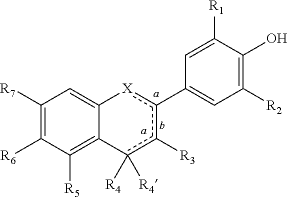

wherein:
X is O when a is a single bond or O⁺ when a is a double bond,
R1 and R2 are independently of each other a hydrogen atom, a hydroxyl group or a methoxyl group,
R3, R5 and R7 are independently of each other, a hydrogen atom, a hydroxyl group (OH), an O-glycosyl group, a (C1-C18) alkoxyl or a group having the formula (II):

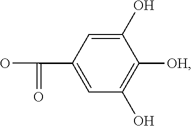 [SMILES: O=C(O)c1cc(O)c(O)c(O)c1]

R4 and R6 are independently of each other, a hydrogen atom or an OH group,
R4′ is a hydrogen atom or R4′ is with R4 and the carbon atom to which they are bound, a C═O group, when a is a single bond or R4′ is nothing when a is a double bond,
a and b are identical or different, being either a single bond or a double bond,

provided that at least one of the R3, R5 and R7 groups is a group having the formula (II) and/or R1 and R2 are both hydroxyl (OH) groups, or one of the pharmaceutically acceptable salts or esters thereof, said compound having the formula (I) being in the form of a pure stereoisomer or in the form of a mixture of enantiomers and/or diastereomers, including racemic mixtures, for use as an antiviral agent in the treatment and/or prevention of infection by the hepatitis C virus (HCV).

Based on the compound having the formula (I), a and b are either a single bond or a double bond and a and b are not simultaneously a double bond.

As used herein, the term “pharmaceutically acceptable” and the grammatical variations relating thereto, refer to compositions, carriers, diluents and reagents, that are used in an interchangeable manner and can be administered to a mammal without inducing adverse physiological effects such as nausea, dizziness, gastric disorders, etc.

The term “pharmaceutically acceptable esters or salts” makes reference to inorganic and organic, relatively nontoxic acid addition salts, or to esters of the compounds of the present invention which are generally prepared by reacting the free acid with an appropriate organic or inorganic base. Such salts or esters may be prepared in situ during the final isolation and purification of the compounds. In particular, acid addition salts may be prepared by separately reacting the purified compound in its purified form with an organic or inorganic acid and by isolating the salt thus formed. Figuring amongst the examples of acid addition salts are hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulfamates, malonates, salicylates, propionates, methylene-bis-b-hydroxynaphthoates, gentisic acid, isethionates, di-p-toluoyltartrates, methanesulfonates, ethanesulfonates, benzenesulfonates, p-toluenesulfonates, cyclohexyl sulfamates and quinateslaurylsulfonate salts, and the like. (See for example S M Berge et al “Pharmaceutical Salts” J Pharm Sci, 66:p 1-19 (1977) which is incorporated herein by reference).

The compound according to the invention is a specific inhibitor of HCV in that within the single stranded RNA virus, it inhibits only one single member of the family Flaviviridae: the HCV. Moreover, unlike a number of anti-HCV viral inhibitors, the compound according to the invention has an effective antiviral effect vis a vis all of the genotypes of HCV and this is so even with respect to the genotypes considered most resistant to conventional antivirals. This holds true in the case of HCV genotype 1. This is what makes it an inhibitor of interest.

In addition, the inventors have shown that among the molecules of the flavonoid family, only molecules with a 3,4,5-trihydroxy-phenyl group had an antiviral effect on HCV. Thus, the presence of the 3,4,5-trihydroxy-phenyl group is essential for the antiviral activity of the compound according to the invention. This structure is found in the galloyl group having the formula (II) as well as in C2 of the central heterocyclic ring (chroman heterocycle) when R1 and R2 are hydroxyl groups.

Based on one variant of the compound according to the invention, any one of the R3, R5 or R7 groups is a group having the formula (II), and the R1 and R2 groups are both OH groups.

Based on a second variant of the compound according to the invention, at least two of the R3, R5 and R7 groups are groups having the formula (II).

The inventors have demonstrated an additive effect of the 3,4,5-trihydroxy-phenyl groups on the antiviral properties of the compounds having the formula (I), such that the antiviral effect of a compound having formula (I) comprising of two 3,4,5-trihydroxy-phenyl groups is much higher than that of a compound having only one group.

Advantageously, the compound according to the invention is a compound having the formula (I) wherein the R3, R5 and R7 groups are independently of one another, an O-alkyl group selected from the group consisting of an O-methyl, O-ethyl or O-propyl group.

The term “O-glycosyl group” is understood to mean one or more carbon chains comprising at least one saccharide, that is to say, a monosaccharide or an oligosaccharide bound to an OH group of the compound having the formula (I), the monosaccharide or oligosaccharide being likely to be substituted by C1-C6 alkyl groups, or by phenolic groups.

Typically the O-glycosyl group comprises at least one monosaccharide selected from the group consisting of glucose, galactose, rhamnose and arabinose. Preferably, the glycosyl group is selected from the group consisting of a monosaccharide, a disaccharide or a trisaccharide.

Advantageously, the compound according to the invention is aglycane.

The invention also relates to a compound having the formula (I) wherein,

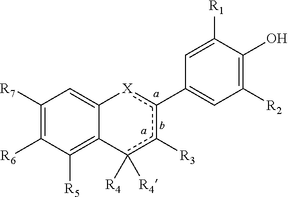

X is O when a is a single bond or O+ when a is a double bond, a and b are identical or different, being either a single bond or a double bond,
the R1, R5 and R7 groups are hydroxyl groups,
R2 is a hydroxyl group or a hydrogen atom,
R3 is a hydroxyl group, a hydrogen atom or a galloyl group having the formula (II)

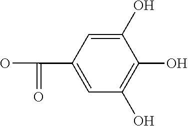 [SMILES: O=C(O)c1cc(O)c(O)c(O)c1]

R4′ is a hydrogen atom when a is a single bond or R4′ is nothing when a is a double bond, and
R6 is a hydrogen atom

or one of the pharmaceutically acceptable salts or esters thereof.

According to a first variant, the compound according to the invention is a compound having the formula (III):

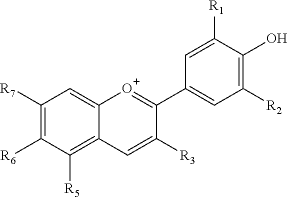

wherein:
R1 and R2 are independently of each other a hydrogen atom, a hydroxyl group or a methoxyl group,
R3, R5 and R7 are independently of each other, a hydrogen atom, an OH group, an O-glycosyl group, a (C1-C18) alkoxyl or a group having the formula (II)

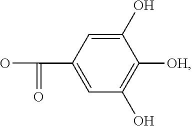 [SMILES: O=C(O)c1cc(O)c(O)c(O)c1]

R6 is a hydrogen atom or an OH group, and

at least one of the R3, R5 or R7 groups is a group having the formula (II), or R1 and R2 are both OH groups,

or one of the pharmaceutically acceptable salts or esters thereof.

Preferably, the compound is an anthocyanidin or anthocyanin. Typically, the compound according to the invention is a compound having the formula (III) wherein R1, R2, R3, R5 and R7 are OH groups and R6 is a hydrogen atom. Such a compound having the formula (III) is Delphinidin (3,3′,4′,5,5′,7-Hexahydroxyflavylium chloride; CAS No 528-53-0). Delphinidin may be modified by glycosylation on the R3, R5 and/or R7 groups. Delphinidin may be in the form of a salt, such as delphinidin chloride.

Typically, the compound according to the invention is the compound having the formula (III) wherein R1, R2, R5 and R7 are OH groups and R3 and R6 are hydrogen atoms. Such a compound having the formula (III) is Tricetinidin (3′,4′,5,5′,7-pentahydroxyflavylium chloride; CAS No 65618-21-5). The tricetinidin may be modified by glycosylation on the R3, R5 or R7 groups. Tricetinidin is found in tea and would be the product of degradation by oxidative degallation of epigallocatechin gallate EGCG.

Typically, the compound according to the invention is the compound having the formula (III) wherein R1, R2, R3 and R7 are OH groups; R6 is a hydrogen atom, R5 is an O-methyl group. Such a compound having the formula (III) is Pulchellidin (3,7,3′,4′,5′-Pentahydroxy-5-methoxyflavylium, CAS No 19077-86-2. Pulchellidin may be modified by glycosylation on the R3 and R7 groups.

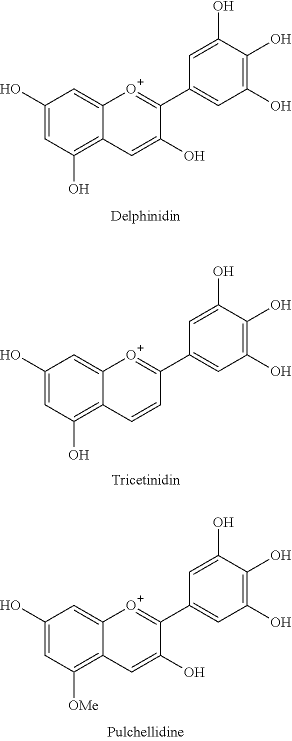 [SMILES: Oc1cc(O)c2cc(O)c(-c3cc(O)c(O)c(O)c3)[o+]c2c1 ; Oc1cc(O)c2ccc(-c3cc(O)c(O)c(O)c3)[o+]c2c1 ; COc1cc(O)cc2[o+]c(-c3cc(O)c(O)c(O)c3)c(O)cc12]

Based on a second variant, the compound according to the invention is a compound having the formula (IV)

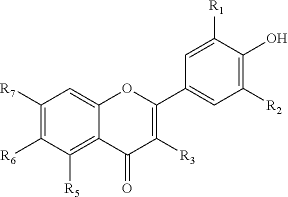

wherein:
R1 and R2 are independently of each other a hydrogen atom, a hydroxyl group or a methoxyl group,
R3 is an OH group, an O-glycosyl group, a (C1-C18) alkoxyl or a group having the formula (II)

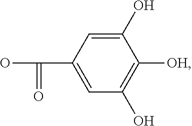 [SMILES: O=C(O)c1cc(O)c(O)c(O)c1]

R5 and R7 are independently of each other, a hydrogen atom, an OH group, an O-glycosyl group, a (C1-C18) alkoxyl or a group having the formula (II),
R6 is a hydrogen atom or an OH group, and

at least one of the R3, R5 or R7 groups is a group having the formula (II), or R1 and R2 are both OH groups,

or one of the pharmaceutically acceptable salts or esters thereof.

Preferably, the compound according to the invention is a Flavonol (3-hydroxy-2-phenylchromen-4-one).

Typically, the compound according to the invention is the compound having the formula (IV) wherein R1, R2, R3, R5 and R7 are OH groups; R6 is a hydrogen atom. Such a compound having the formula (IV) is Myricetin (or 3,3′,4′,5′,5,7-hexahydroxy-2-phenylchromen-4-one; CAS No 529-44-2). Myricetin may be modified by glycoylation in particular on the R3 group.

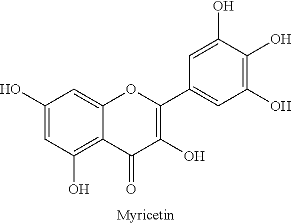 [SMILES: O=c1c(O)c(-c2cc(O)c(O)c(O)c2)oc2cc(O)cc(O)c12]

According to a third variant, the compound according to the invention is a compound having the formula (V)

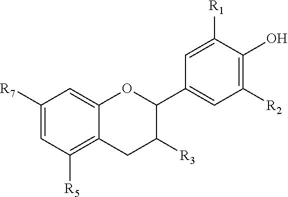

wherein:
R1 and R2 are independently of each other a hydrogen atom, a hydroxyl group or a methoxyl group,
R3 is an OH group, an O-glycosyl group, a (C1-C18) alkoxyl or a group having the formula (II)

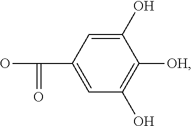 [SMILES: O=C(O)c1cc(O)c(O)c(O)c1]

R5 and R7 are independently of each other, a hydrogen atom, an OH group, an O-glycosyl group, a (C1-C18) alkoxyl or a group having the formula (II), and

at least one of the R3, R5 or R7 groups is a group having the formula (II), or R1 and R2 are both OH groups,

or one of the pharmaceutically acceptable salts or esters thereof.

Preferably, the compound according to the invention is a flavan-3-ol (flavanol or catechin).

Catechins are a subfamily of flavonoids whose structure is based on the 2-phenyl-3-chromano.

Typically, the compound according to the invention is the compound having the formula (V) wherein R1, R2, R3, R5 and R7 are OH groups, the compound according to the invention is referred to as (−)-Epigallocatechin (EGC) ((2R,3R)-2-(3,4,5-trihydroxyphenyl) chroman-3,5,7-triol; CAS No 970-74-1) when the C2 and C3 groups of the central heterocyclic ring (chroman heterocycle) are in cis position and referred to as (−)-Gallocatechin (2S,3R)-3,4-dihydro-2-(3,4,5-trihydroxyphenyl)-2H-1-benzopyran-3,5,7-triol; CAS No 3371-27-5) or ((+)-Gallocatechin (GC) (2R,3S)-2-(3,4,5-Trihydroxyphenyl)-3,4-dihydro-2H-1-benzopyran-3,5,7-triol; CAS No 970-73-0) when the C2 and C3 groups of the central heterocyclic ring (chroman heterocycle) are in trans position.

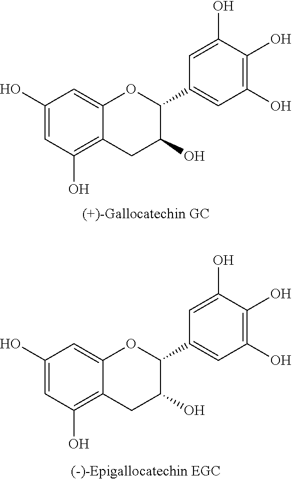 [SMILES: Oc1cc(O)c2c(c1)O[C@H](c1cc(O)c(O)c(O)c1)[C@@H](O)C2 ; Oc1cc(O)c2c(c1)O[C@H](c1cc(O)c(O)c(O)c1)[C@H](O)C2]

Typically, the compound according to the invention is the compound having the formula (V) wherein R1, R5 and R7 are OH groups; R2 is a hydrogen atom; R3 is a galloyl group having the formula (II), the compound according to the invention being known as (−)-Epicatechin-3-gallate (ECG) ((2R,3R)-2-(3,4-Dihydroxyphenyl)-3,4-dihydro-1(2H)-benzopyran-3,5,7-triol 3-(3,4,5-trihydroxybenzoate); CAS No 1257-08-5) when the C2 and C3 groups of the central heterocyclic ring (chroman heterocycle) are in cis position and referred to as catechin-3-gallate (CG); ((2S,3R)-2-(3,4-Dihydroxyphenyl)-3,4-dihydro-1(2H)-benzopyran-3,5,7-triol 3-(3,4,5-trihydroxybenzoate); CAS No 130405-40-2) when the C2 and C3 groups of the central heterocyclic ring (chroman heterocycle) are in trans position.

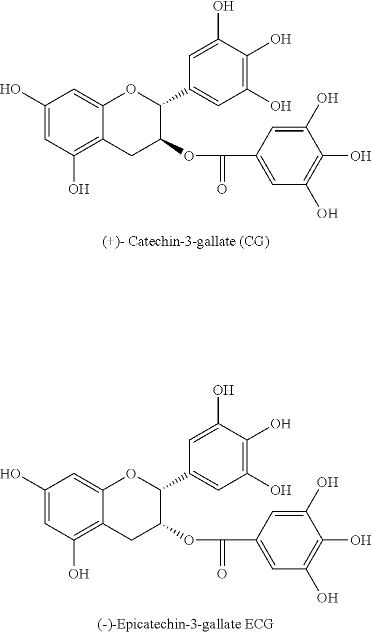 [SMILES: O=C(O[C@H]1Cc2c(O)cc(O)cc2O[C@@H]1c1cc(O)c(O)c(O)c1)c1cc(O)c(O)c(O)c1 ; O=C(O[C@@H]1Cc2c(O)cc(O)cc2O[C@@H]1c1cc(O)c(O)c(O)c1)c1cc(O)c(O)c(O)c1]

Typically, the compound according to the invention is the compound having the formula (V) wherein R1, R2, R5 and R7 are OH groups; R3 is a galloyl group having the formula (II), the compound according to the invention being known as (−)-Epigallocatechin-3-gallate (EGCG) ((2R,3R)-5,7-dihydroxy-2-(3,4,5-trihydroxyphenyl)-3,4-dihydro-2H-chromen-3-yl 3,4,5-trihydroxybenzoate; CAS No 989-51-5) when the C2 and C3 groups of the central heterocyclic ring (chroman heterocycle) are in cis position and referred to as (−)-Gallocatechin gallate (GCG) ((2S,3R)-2-(3,4,5-trihydroxyphenyl)-3,4-dihydro-1(2H)-benzopyran-3,5,7-triol 3-(3,4,5-trihydroxybenzoate). CAS No 4233-96-9) when the C2 and C3 groups of the central heterocyclic ring (chroman heterocycle) are in trans position.

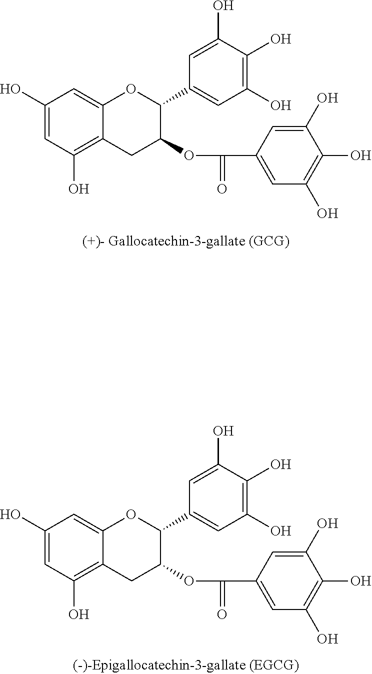 [SMILES: O=C(O[C@H]1Cc2c(O)cc(O)cc2O[C@@H]1c1cc(O)c(O)c(O)c1)c1cc(O)c(O)c(O)c1 ; O=C(O[C@@H]1Cc2c(O)cc(O)cc2O[C@@H]1c1cc(O)c(O)c(O)c1)c1cc(O)c(O)c(O)c1]

Preferably, the compound according to the invention is selected from the group consisting of Delphinidin, Myricetin, Tricetinidin, Pulchellidin. Epigallocatechin (EGC), Epigallocatechin Gallate (EGCG), Gallocatechin (GC), Catechin Gallate (CG), Gallocatechin Gallate (GCG), Epicatechin Gallate (ECG), the pharmaceutically acceptable salts and esters thereof, and mixtures thereof.

In an advantageous manner, said compound according to the invention is selected from the group consisting of Epigallocatechin (EGC), Epigallocatechin Gallate (EGCG), Gallocatechin (GC), Catechin Gallate (CG), Gallocatechin Gallate (GCG), Epicatechin Gallate (ECG), the pharmaceutically acceptable salts and esters thereof, and mixtures thereof.

Preferably, said compound according to the invention is Epigallocatechin Gallate (EGCG) or one of the pharmaceutically acceptable salts or esters thereof.

The flavonoid compounds according to the invention being substances occurring naturally in plants and especially in most fruits and vegetables, they may be obtained by purification. By way of example, catechins may be obtained by extraction from green or black tea through various filtration processes, such as those described in the U.S. Pat. No. 6,383,392 or EP 1 077 211. Anthocyanins may also be obtained by purification from plants as described in the state of the art documents and in particular in the patent applications US 2010041877 or US 2003147980. Such flavonoid compounds may be found in commercial channels.

The flavonoid compounds may also be obtained by means of chemical synthesis by implementing methods known to the person skilled in the art such as in particular solid phase synthesis methods. Such flavonoid compounds may be found in commercial channels.

In the context of the present invention, the term “Hepatitis C virus” or “HCV” should be understood as referring to all genotypes of the hepatitis C virus, genotypes that may or may not already be described, in particular genotypes numbered 1 to 6, as well as their subtypes, especially genotypes 1a, 1b, 2a, 2b, 3, 4, 5, and 6. This term should also be understood as referring to a recombinant or non recombinant HCV virus capable of infecting a host, such as a target cell of a subject. The terms “target cell(s)” or “HCV target cell(s)”, “host cell(s)” or “HCV host cell(s)” should be understood as referring to cells susceptible to being infected with HCV. The host cell or target cell according to the present invention is the hepatocyte.

The term “antiviral agent” is used to refer to an agent that is capable of interacting directly with the HCV virus or with one or more of its constituents and induces inhibition of HCV infection either through the reduction or abolition of viral entry, viral replication or pathogenesis or any other event occurring in the host cell or combinations thereof. This direct action of the antiviral agent is in marked contrast to the indirect action of the compounds used in the treatment of HCV infections and acting by stimulating the immune system, for example.

The term “infection by the HCV virus” or “viral infection” in reference to HCV, refers to the condition of a subject or a patient infected by the HCV virus. Infection by the HCV virus refers to either an asymptomatic or chronic HCV infection, regardless of their stage of development. This term also refers to the entry, replication or any other event or process involved in the pathogenesis of the HCV virus in a host cell. Thus, the infection comprises the introduction of an infectious agent, such as a recombinant or non recombinant HCV virus capable of infecting a host, such as a target cell of a subject.

The term “prevention”, as currently used in reference to an HCV infection is related to the reduction of risk or to inhibition of the development of a viral infection. The compound according to the invention being able to inhibit the early stages of infection is particularly advantageous in the prevention of HCV infection.

The term “treatment” as used currently, generally refers to the improvement of a sign or symptom of a disease or a pathological condition related, for example, to the HCV infection in particular due to the inhibition of the virus, stopping of its development, the decrease in viral load of the patient or the eradication of the virus. Reference is thus made to the means of bringing about the regression, reduction, inhibition of the progression of HCV, or the prevention of HCV infection or one or more symptoms of this infection.

The said compound according to the invention is an antiviral agent in particular, for inhibiting the infection of a target cell (such as a hepatocyte cell) by the hepatitis C virus and/or transmission of the hepatitis C virus between two target cells. For example, from an infected hepatocyte cell to a non-infected hepatocyte cell.

The compound according to the invention inhibits the two major modes of viral infection, that is, the infection of a healthy target cell (such as a non infected hepatocyte cell) by the isolated virus and the second mode, viral dissemination from cell to cell, in other words, infection of a non infected target cell by an infected target cell. This latter mode of transmission is important because it is suspected to be a predominant in vivo pathway for certain families of enveloped viruses.

Preferably, said compound is an antiviral agent for inhibiting the entry of hepatitis C virus. The use of an inhibitor of viral entry may be particularly advantageous in the case of liver transplantation in order to prevent infection of the transplanted liver. The inventors have demonstrated such an inhibition method, by the use of a reference study model allowing the expression of the viral envelope glycoproteins of interest that are infectious virus like particles.

Thus, according to the invention, the term “inhibition of viral entry” or “inhibition of virus entry” should be understood as inhibition of the passage of a viral genome from outside to inside the host cell, whether it involves an infection mediated by an individualised viral particle or any other mechanism leading to infection of a non-infected host cell. This term may also be understood to refer to inhibition of any of the steps of the viral infection prior to release of the virus genome in the cytoplasm of the host cell. These steps preceding release are: i) adhesion or attachment of the virus or an infected host cell to the surface of a second host cell (typically a non infected host cell), ii) interaction with the specific viral receptor(s) and iii) fusion of the viral lipid envelope of the virus particle to a cell membrane (plasmic or endosomal) enabling the introduction of the viral genome into the cytoplasm of the infected cell. The inventors have carried out experiments aimed at splitting up the mechanism of HCV entry into the host cell into its three constituent steps and have thus shown that only the step of attachment is inhibited by the compounds having the formula (I) such as EGCG, ECG, EGC or Delphinidin. The step of attachment is the earliest of the steps involved in the mechanism of viral entry. Such results may be reinforced by experiments pertaining to “binding” and quantification by quantitative RT-PCR of the virus RNA attached to the cell or quantification of the capsid protein.

Preferably, said compound is an antiviral agent for inhibition of surface glycoproteins of HCV in their function during the steps relating to entry of the viral genome into the non infected host cell. This may be confirmed by pull down or microcalorimetry tests and surface plasmon resonance analysis (Biacore).

Advantageously, the compound according to the invention is an antiviral agent for inhibition of the adhesion of the hepatitis C virus, or of a host cell infected with hepatitis C, to the membrane of a non infected host cell.

Preferably, said compound is an antiviral agent for inhibition of the interaction of the surface glycoproteins of HCV with at least one target protein of a host cell, and more particularly inhibition of the glycoproteins of HCV such as the E1 and/or E2 glycoproteins, and in particular, the E1 protein (Acc No: AAB67037 SEQ ID NO: 1) or the E2 protein (Acc No: AAB67037 SEQ ID NO: 2) of HCV of genotype 1a, the E1 protein (Acc No: AY734976 SEQ ID NO: 3) or the E2 protein (Acc No: AY734976 SEQ ID NO: 4) of HCV of genotype 1b, the E1 protein (Acc No: AB047639 SEQ ID NO: 5) or the E2 protein (Acc No: AB047639 SEQ ID NO: 6) of HCV of genotype 2a, the E1 protein (Acc No: AY734982 SEQ ID NO: 7) or the E2 protein (Acc No: AY734982 SEQ ID NO: 8) of HCV of genotype 2b, the E1 protein (Acc No: AY734984 SEQ ID NO: 9) or the E2 protein (Acc No: AY734984 SEQ ID NO: 10) of HCV of genotype 3a, the E1 protein (Acc No: AY734986 SEQ ID NO: 11) or the E2 protein (Acc No: AY734986 SEQ ID NO: 12) of HCV of genotype 4, the E1 protein (Acc No: AY785283 SEQ ID NO: 13) or the E2 protein (Acc No: AY785283 SEQ ID NO: 14) of HCV of genotype 5, the E1 protein (Acc No: AY736194 SEQ ID NO: 15) or the E2 protein (Acc No: AY736194 SEQ ID NO: 16) of HCV of genotype 6.

The term “inhibition of the interaction of the surface glycoproteins of HCV” or “inhibition of the binding of the surface glycoproteins of HCV” or “inhibition of the interaction of the glycoproteins E1 and/or E2 “is understood to mean the inhibition of the specific binding of at least one of the surface glycoproteins of HCV and in particular one of the glycoproteins E1 or E2 with at least one protein or glycoprotein of a target host cell. For example, the inhibition of the binding between the glycoproteins E1 and/or E2 and the glycosaminoglycans of hepatocytes. The surface glycoproteins of HCV may be found on the surface of a viral particle, but also bind to the surface of an infected target cell. Binding of the surface glycoproteins of HCV and in particular glycoproteins E1 and/or E2 to the target proteins takes place during the step of attachment or adhesion of the HCV with a target cell, or the step of attachment or adhesion of an infected target cell to another target cell.

In the context of the present invention, the “target proteins” or “target proteins of a host cell” are the surface proteins or glycoproteins, that is transmembrane proteins or glycoproteins or anchored to the surface of the plasma membrane of host cells, these proteins being specifically recognised by the surface glycoproteins of HCV and in particular the glycoproteins E1 and/or E2. The Glycosaminoglycans of a target cell, such as a hepatocyte may be noted by way of examples.

Thus, the inventors have demonstrated that inhibition by the compounds having the formula (I) such as EGCG, ECG, EGC or Delphinidin specifically involves the glycoproteins E1 and/or E2. The compounds according to the invention inhibit the interaction between the E1 and/or E2 protein of HCV and the target proteins such as the glycosaminoglycans of hepatocytes. In addition, the inventors have clearly demonstrated that the inhibitory effect on viral entry mediated by the compounds having the formula (I) such as EGCG, ECG, EGC or Delphinidin, directly involves the viral envelope glycoproteins in a conserved region thereof. In fact, the inventors have shown that the inhibitory effect of the compounds having the formula (I) such as EGCG, ECG, EGC or Delphinidin was observed in all the viral genotypes of HCV namely the genotypes 1a, 1b, 2a, 2b, 3, 4, 5 and 6 indicating that the binding of molecules according to the invention with the glycoproteins E1 and/or E2 is performed in a highly conserved region of the latter.

According to the invention, the treatment and/or prevention of infection by the hepatitis C virus is intended for a patient who is resistant or intolerant to treatment for an HCV infection, by an immunomodulatory agent and/or an inhibitor of viral metabolism.

The genotype 1 of HCV is the most resistant to current treatments. It is responsible for 70% of known cases of hepatitis C in the United States, Japan and Western Europe. However, less than 50% of patients achieve a sustained virological response with the current treatment. The compound according to the invention provides for the inhibition of infection by the HCV virus of all genotypes described and more particularly HCV of genotype 1 for which it demonstrates a very strong antiviral capacity.

The “viral inhibitors” may be defined under three groups.

The first group includes inhibitors of viral metabolism. The term “inhibitors of viral metabolism” is understood to refer to i) inhibitors of translation in particular Internal Ribosome Entry Site (IRES) inhibitors, ribozymes or siRNAs; ii) inhibitors of protein maturation, such as protease inhibitors (NS3/NS4a protease); iii) inhibitors of the replication of the viral genome in particular inhibitors of the binding between the viral RNA and the polymerase RNA or inhibitors of NS5B polymerase that may be nucleoside or non-nucleoside inhibitors or inhibitors of helicase; vi) inhibitors of viral assembly such as inhibitors of glycosylation.

The second group of inhibitors includes “inhibitors of viral entry” into the hepatocyte. Those considered as such include any molecule capable of inhibiting, either totally or partially, the step of recognition of the host cell by an individual virus particle or by the membrane of an infected host cell or the step of attachment of the virus or the membrane of an infected host cell, to the cell surface of the non infected host cell or the step of endocytosis of the viral particles by the host cell or the step of fusion of the viral membrane with the endosomal membrane.

The third group includes “immunomodulatory agents”. The term “immunomodulatory agents” is understood to refer to molecules of synthetic or natural origin that aid towards initiating or potentiating the immune response in mammals during a viral infection. By way of examples of immunomodulatory agents mention may be made of type 1 interferons (such as the interferons alpha (IFN-α), beta (IFN-β) or omega (IFN-ω)), type 2 interferons (such as gamma interferon IFN-γ) and pegylated interferons.

According to a first preferred variant, the compound according to the invention is co-administered with an additional agent meant for use in the treatment and/or prevention of an HCV infection. Said additional agent is preferably, a viral inhibitor as defined here above.

The use of such a combination of multiple inhibitors makes it possible to reduce the risks related to viral resistance often observed in the context of treatment of viruses with high mutation rates.

Advantageously, the additional agent is an immunomodulatory agent or an inhibitor of the viral metabolism of HCV.

According to a second preferred variant, said compound is the only antiviral agent administered in the treatment and/or prevention of infection with the hepatitis C virus.

Advantageously, said compound is formulated in the form of a pharmaceutical composition. Typically, said pharmaceutical composition contains a pharmaceutically acceptable carrier and the compound according to the invention.

The term “pharmaceutically acceptable carrier” is understood to refer to any solvent, dispersion medium, absorption delaying agent, etc, which does not produce secondary reactions, for example allergic reactions, in humans or animals. The pharmaceutically acceptable carriers are well known to persons skilled in the art and include those described in “Remington's Pharmaceutical Sciences” (Mack Publishing Company, Easton, USA, 1985). The particular pharmaceutically acceptable carrier is selected in particular depending on the route of administration, which may for example be oral, sublingual, nasal, buccal, transdermal, intravenous, subcutaneous, intramuscular and/or rectal. The dose depends on factors such as the active ingredient in question, the mode of administration, the therapeutic indication, age, weight and condition of the patient.

The invention also relates to a method of treatment or prevention of HCV infection, comprising the administration to an individual in need thereof of a therapeutically effective amount of a compound having the formula (I), the pharmaceutically acceptable salts and esters thereof or mixtures thereof, said compound having the formula (I) being in the form of pure stereoisomers or in the form of mixtures of enantiomers and/or diastereomers, including racemic mixtures, preferably, said compound having the formula (I) is selected from the group consisting of Delphinidin, Myricetin, Tricetinidin, Pulchellidin, Epigallocatechin (EGC), Epigallocatechin Gallate (EGCG), Gallocatechin (GC), Catechin Gallate (CG), Gallocatechin Gallate (GCG), Epicatechin Gallate (ECG), the pharmaceutically acceptable salts and esters thereof, and mixtures thereof. The individual is preferably a mammal, more particularly a human. The therapeutically effective amount may be easily determined by the person skilled in the art.

The term “therapeutically effective amount” or “therapeutically effective dose” as used herein refers to the amount of antiviral agents such as flavonoid compounds having the formula (I) or their derivatives that cause a biological or medicinal response in a tissue, an animal or human biological system, which includes alleviation of the symptoms of HCV infection mainly due to the inhibition of the virus, the stopping of its development, the decrease in viral load of the patient or the eradication of the virus. For the purposes of prevention, a therapeutically effective amount may also be considered as a “prophylactic amount” of active agents.

The terms “patient” or “patient in need thereof” are understood to refer to a human or non-human mammal affected or likely to be affected by HCV. In a preferred embodiment, the patient is a human. In another embodiment, the patient is a chimpanzee.

The object of the invention also relates to a method preferentially ex vivo for reduction of the infectivity or inactivation of a hepatitis C virus (HCV) comprising a step of contacting said hepatitis C virus with a compound having the formula (I):

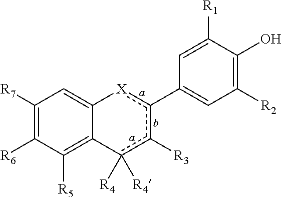

wherein:
X is O when a is a single bond or O⁺ when a is a double bond,
R1 and R2 are independently of each other a hydrogen atom, a hydroxyl group or a methoxyl group,
R3, R5 and R7 are independently of each other, a hydrogen atom, an OH group, an O-glycosyl group, a (C1-C18) alkoxyl or a group having the formula (II):

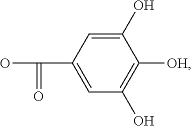 [SMILES: O=C(O)c1cc(O)c(O)c(O)c1]

R4 and R6 are independently of each other, a hydrogen atom or an OH group,
R4′ is a hydrogen atom or R4′ is with R4 and the carbon atom to which they are bound, a C═O group, when a is a single bond or R4′ is nothing when a is a double bond,
a and b are identical or different, being either a single bond or a double bond,

provided that at least one of the R3, R5 and R7 groups is a group having the formula (II) and/or R1 and R2 are both OH groups,

or one of the pharmaceutically acceptable salts or esters thereof, said compound having the formula (I) being in the form of a pure stereoisomer or in the form of a mixture of enantiomers and/or diastereomers, including racemic mixtures.

The HCV is contacted with the compound having the formula (I) for a period of time and under conditions that are sufficient to achieve the inactivation or the inhibition either partial or complete of the virus. According to the invention, the HCV is contacted with the compound having the formula (I) before or during the step of infection. The step of infection is the step of contacting the virus with the target cell or cells thereof.

The term “infectivity” is understood to refer to the infectious nature of the virus, that is the capacity of the virus to bring about a viral infection within the meaning of the present invention, in particular the reduction in the ability of the virus to enter into a target cell, preferentially the reduction in its ability to attach to a target cell.

Advantageously, the ex vivo method according to the invention may be implemented by contacting a sample which is likely to contain an HCV virus and a compound having the formula (I) in liquid or semi liquid medium, the compound having the formula (I) is preferably applied at a concentration of 0.5 μM to 100 μM, advantageously at a concentration of 10 μM to 80 μM or from 20 μM to 70 μM. Typically, the compound having the formula (I) is applied at a concentration of 50 μM. The method may be implemented at a temperature of 40° C. to 37° C., preferably from 4° C. to 10° C. or from 20° C. to 37° C.

The compound according to the invention may be contacted with said virus for a period of 15 to 90 min, preferably, from 30 to 45 min.

Preferably, according to the ex vivo method for reduction or inactivation of the infectivity of the HCV, said hepatitis C virus is present in a biological sample.

The term “biological sample” refers to a sample of biological origin or obtained from a biological organism or entity such as an organ, a tissue, a cell, a population of cells, a biological fluid, a purified protein or peptide. Without limitation thereof examples include liquid samples of biological origin such as blood, plasma, serum, cerebrospinal fluid, lymph or cell lysates; solid samples such as organs in particular intended for transplantation use or tissues or cell cultures that may be intended for graft related use.

According to an advantageous variant, the ex vivo method of reduction of infectivity or inactivation of HCV according to the invention comprises an additional step of washing of the biological sample. This step allows the reduction or elimination of the compound having the formula (I) from said biological sample. The step of washing may be preceded by a step of incubation of the mixture of compound having the formula (I) in said biological sample.

The ex vivo method according to the invention may also comprise an additional step of checking and monitoring the infectivity of a biological sample. Such a step may be considered before the step of contacting the compound having the formula (I) with the biological sample or after the contacting, in order to determine if another cycle of contacting the biological sample with the compound having the formula (I) should be considered. Such a step of checking and monitoring may be performed by any means enabling in particular, the early detection of an HCV infection. Thus, the detection of a specific marker of HCV can be carried out, for example by the use of a viral protein specific antibody or by detection using PCR analysis of the HCV RNA.

### DESCRIPTION OF FIGURES

FIG. 1: Illustration of the relative viral infection of Huh-7 cells with the JFH1-Rluc virus based on the presence of (+)-catechin (C), (−)-epicatechin (EC), (−)-epicatechin-3-gallate (ECG), (−)-epigallocatechin (EGC) and (−)-epigallocatechin-3-gallate marketed by EXTRASYNTHESE® (EGCG EXTRASYNTHESE®) or by CALBIOCHEM® (EGCG-CALBIOCHEM®) at 50 μM, or DMSO.

FIG. 2: Illustration of the relative viral infection of Huh-7 cells by the JFH1-Rluc virus based on the EGCG concentration (0; 0.5; ; 5; 50 μM) in the medium during infection or pretreatment of the virus with 50 μM of EGCG before the infection step.

FIG. 3: Illustration of the relative viral infection of Huh-7 cells infected with infectious virus like particles (HCVpp) expressing on their surface the envelope proteins E1 and E2 of the HCV virus of genotypes 1a, 1b, 2a, 2b, 3, 4, 5, or 6 or infected with virus like particles of the vesicular stomatitis virus VSV expressing the envelope protein of VSV (VSVpp) based on the presence of EGCG (50 μM) or Dimethyl Sulfoxide (DMSO) in the infection medium.

FIG. 4: Illustration of the relative viral infection of Huh-7 cells infected with the yellow fever virus (YFV) or the Sindbis virus (SINV) or the Mardin Darby Bovine Kidney (MDBK) cells infected with the bovine viral diarrhoea virus (BVDV) based the presence of EGCG (50 μM) or DMSO in the infection medium.

FIG. 5: Illustration of the number of cells per infection site for the viral infection of Huh-7 cells with the JFH1 virus in the presence or absence of EGCG (at 50 μM).

FIG. 6: Illustration of the percentage of cells infected with cell supernatants during passages P0 to P4 subsequent to the infection medium being treated with DMSO or EGCG.

FIG. 7: FIG. 7 A. Diagram illustrating the experimental conditions for the study of the inhibitory effect of EGCG on the entry of HCV (Sample 5) and more particularly on the steps of attachment (Sample 2), interaction with receptors of the host cell (Sample 3) and the step of endocytosis and fusion of viral and cellular membranes (Sample 4), DMSO was used as a control (Sample 1).

FIG. 7B. Illustration of the relative viral infection of Huh-7 cells by the JFH1-Rluc virus based on the presence of DMSO or EGCG (at 50 μM) in samples 1 to 5.

FIG. 8: Illustration of the relative viral infection of Huh-7 cells by the HCVcc virus based on the presence of EGCG or Delphinidin (at increasing concentrations). This graph shows an experiment representative of three experiments carried out independently.

FIG. 9: Illustration of the relative viral attachment of the HCVcc virus to Huh7 cells based on the presence of DMSO, 50 μM of EGCG, 50 μM of Delphinidin Chloride or 500 μg/mL of Heparin. The relative attachment is expressed as a percentage of the control (DMSO) for which a value of 100% has been arbitrarily assigned.

### EXAMPLES

### Materials and Methods

### Reagents.

The Dulbecco Modified Medium (Dulbecco's modified Eagle's medium DMEM, GIBCO), the Dulbecco Minimum Essential Medium (Minimum Essential Medium Eagle MEM, GIBCO®), the saline phosphate buffer (phosphate-buffered saline PBS), the reduced serum medium (GIBCO® OptiMEM®), L-alanyl-L-glutamine (GLUTAMAX-I™), goat serum, horse serum as well as foetal calf serum (FCS) are products marketed by INVITROGEN®. The 4′,6-Diamidino-2-phenylindole (DAPI) was obtained from MOLECULAR PROBES®. The (−)-Epigallocatechin Gallate (EGCG CALBIOCHEM®) and polyvinyl alcohol (MOWIOL® 3-88) are marketed by CALBIOCHEM®. The in vivo transfection reagent ExGen500® is marketed by EUROMEDEX®. The (+)-Catechin, (−)-Epicatechin, (−)-Epicatechin-3-Gallate (ECG), (−)-Epigallocatechin (EGC) and (−)-Epigallocatechin-3-Gallate (EGCG Extrasynthèse®) are marketed by the company Extrasynthèse® (Lyon, France). The Delphinidin Chloride is produced by Extrasynthèse (Lyon, France). The other reagents are marketed by the company SIGMA®.

### Antibodies.

The mouse monoclonal antibodies (MAbs) directed against the envelope proteins (E) of the yellow fever virus (YFV) referenced as MAb 2D12 (ATCC CRL-1689) and the mouse monoclonal antibodies directed against the NS3 protein of the bovine viral diarrhoea virus (anti-BVDV) referenced as NS3 MAb Osc-23 (Boulanger, D, et al, J Gen Virol, 1991. 7: p. 1195-1198) were produced in vitro by using the MiniPerm® (HERAEUS®) apparatus in accordance with the manufacturer's recommendations. The mouse monoclonal antibodies anti-CD81 referenced as MAb 5A6 (Oren. R, et al, Mol Cell Biol, 1990. 10(8): p 4007-15) were kindly provided by S Levy (Stanford University) and the monoclonal antibodies anti-CD81 (referenced as MAb JS-81) are marketed by BD Pharmingen®. The Cy3 goat anti-mouse IgG antibody conjugates are marketed by MOLECULAR PROBES®.

### Cell Lines and Cell Culture Conditions

The human hepatic cell line Huh-7 (Nakabayashi, H et al; Cancer Res. 1982. 42(9) p 3858-63) as well as the HEK 293T cells were cultured in DMEM medium (Invitrogen®) supplemented with L-alanyl-L-glutamine (GLUTAMAX-I™) and 10% foetal calf serum. The Madin-Darby bovine kidney cells (Madin-Darby Bovine Kidney MDBK) were cultured in DMEM medium supplemented with L-alanyl-L-glutamine (GLUTAMAX-I™) with 10% horse serum. The BHK-21 cells were cultured in MEM medium (Invitrogen®) supplemented with L-alanyl-L-glutamine (GLUTAMAX-I™) and with 10% foetal calf serum.

### HCVcc.

The modified version of the plasmid encoding the genome of the isolate JFH1 (genotype 2a; GenBank accession number AB237837) was kindly provided by Mr T Wakita (National Institute of Infectious Diseases, Tokyo, Japan) (Wakita. T, et al. Nat. Med. 2005. 11(7): p 791-6). The modified JFH1 viral clone contains mutations leading to the following amino acid changes F172C, and P173S N534K which have been shown to induce an increase in viral load (Delgrange, D. et al, J Gen Virol. 2007. 88(Pt 9): p 2495-503). In addition, the E1 sequence encoding 196TSSSYMVTNDC residues has been modified so as to reconstruct the epitope A4 (SSGLYHVTNDC) of the E1 glycoprotein of HCV (Dubuisson, J, et al, J Virol, 1994. 68(10): p 6147-60) as described in the state of the art literature (Goueslain. L, et al, J Virol, 2010. 84 (2): p 773-87). The plasmid JFH-Luc contains the gene for Renilla reniformis luciferase (Rluc) as reporter gene and the plasmid JFH-ΔE1E2-Luc contains a deletion that does not lead to a shift in the reading frame within the E1E2 region (Wakita, 7; et al. Nat Med, 2005. 11(7): p 791-6), as described in the prior art (Goueslain, L, et al, J Virol, 2010. 84 (2): p 773-87 and Rocha-Perugini, V, et al, PLoS ONE, 2008. 3(4): p e1866).

In order to generate a genomic RNA of HCV, the plasmids were linearised at the 3′ end of the HCV cDNA by a cut at the XbaI restriction site. Consecutively to treatment with Mung bean nuclease, the linear DNA is then used as a template for in vitro transcription with the MEGAscript® kit marketed by AMBION®. The RNA transcribed in vitro was transfected by electroporation into the Huh-7 cells as described in the prior art (Kato, 7; et al, Gastroenterology. 2003. 125(6): p 1808-1817). The viral extracts were obtained as described in the prior art (Delgrange, D. et al, J Gen Virol, 2007. 88(Pt 9): p 2495-503).

With regard to the tests for infection with the HCVcc viruses, the Huh-7 cells were cultured in 24 well culture plates, in which the infection was performed over a period of 2 hours at 37° C. With regard to the tests with EGCG, the viruses were preincubated in the presence of EGCG for 45 min prior to infection and/or with addition of EGCG in the course of infection with the culture medium until the end of the reaction, unless otherwise indicated. During certain tests, EGCG is added after the step of infection over varying time periods (24, 48 or 72 hours post-infection). The rate of infection is assessed by measuring the luciferase activity 48 hours after the step of infection in cell lysates, by using the Renilla luciferase assay System kit marketed by PROMEGA® or by immunofluorescence with anti-E1 antibodies (A4), 48 hours or 72 hours after infection.

### HCV Pseudoparticles (HCVpp).

Pseudotyped retroviral particles have been described in the prior art (Bartosch. B, J Dubuisson and F L, J Exp Med 2003. 197(5): p 633-42). In brief, 2931′ cells were co-transfected with a transfection vector derived from the murine leukemia virus (MLV) encoding luciferase (Op De Beeck A, et al, J Virol, 2004. 78(6): p 2994-3002), a vector comprising a construct of Gag-Pol genes of the murine leukemia virus and a vector expressing the envelope glycoprotein. The co-transfection had been implemented using the transfection reagent Exgen® 500 under the conditions recommended by the supplier. The following plasmids encoding the viral envelope glycoprotein of HCV of genotype 1b—UKN1b-5.23 (AY734976); of genotype 2b—UKN2b-1.1 (AY734982); of genotype 3a—UKN3a-1.28 (AY734984); of genotype 4—UKN4-11.1 (AY734986); of genotype 5—UKN5-14.4 (AY785283); of genotype 6—UKN6-5.340 (AY736194), were kindly provided by J Ball (University of Nottingham, UK) (Lavillette, D, et al, Hepatology, 2005. 41(2): p 265-74). The plasmid of genotype 1a-H77 (AAB67037 with a mutation modifying 3 amino acids at: R564c, V566A, G650E) has been described in the prior art (Bartosch, B, J Dubuisson and F L, J Exp Med 2003. 197(5): p 633-42), and the plasmid of genotype 2a-JFH-1 (AB047639) was kindly provided by R. Bartenschlager (University of Heidelberg, Germany). The expression vector phCMV-G encoding the G protein of vesicular stomatitis virus (VSV G), has been used for the controlled production of pseudotyped retroviral particles bearing the VSV G envelope glycoproteins on centres corresponding to the murine leukemia virus (VSVpp). Transfection reactions by HCVpp expressing luciferase have been described in the literature (Op De Beeck, A, et al, J Virol, 2004. 78(6): p 2994-3002).

### Other Viruses

The NADL strain of the bovine viral diarrhoea virus (BVDV) and the 171) strain of yellow fever virus (YFV) were used as controls. The BVDVs were produced as previously described (Lecot. S, et al, J Virol 2005. 79(16): p 10826-9). The MDBK cells were seeded on the coverslips in 24 well plates and infected 24 hours later. The cells are infected in the presence of EGCG for 1 hour at 37° C. with BVDV at multiplicity of infection (MI) of about 1, then they are cultured for 15 hours. The infected cells as well as the control cells were rinsed in PBS buffer and then fixed with 3% paraformaldehyde, in order to undergo an immunofluorescence staining with antibody anti-NS3 MAb (monoclonal antibody) (Osc-23). With regard to the YFV infections, the Huh-7 cells were seeded on coverslips in a 24 well plate and infected 24 hours later. The cells were infected in the presence of EGCG for 1 hour at 37° C. with the YFV virus and at MI of about 1, and then they were cultured for 23 hours. The infected cells as well as the control cells were rinsed in the PBS buffer, fixed with 3% paraformaldehyde. The immunofluorescence reaction is conducted with an antibody anti-E MAb 2D12. The Toto 1101/Luc samples (Bick, M. J. et al, J Virol 2003. 77(21): p 11555-62) as well as the Sindbis virus (SINV) expressing luciferase (Firefly luciferase system) (kindly provided by Mr MacDonald, Rockefeller University, NY, USA) were obtained by electroporation of BHK-21 cells from RNA transcribed in vitro. In brief, 15 μg of RNA were mixed with 4×106 BHK-21 cells followed by a step of electroporation at 25 μF and 140V with a square wave electroporator. The supernatant was recovered after 48 hours.

### Indirect Immunofluorescence Microscopy.

The procedure for detection of immunofluorescent viral proteins expressed in infected cells is implemented as described in the literature (Rouille, Y, et al. J Virol, 2006. 80(6): p 2832-41). The nuclei are stained by a 5 min incubation in a PBS buffer containing 1 μg/ml of 4′,6′-diamidino-2-phenylindole (DAPI). The coverslips are mounted on glass slides using mounting medium Mowiol® (10% Mowiol, 25% glycerol, 0.1 M Tris-HCl pH 8.5) and then observed by means of a ZEISS® AXIOPHOT fluorescence microscope equipped with magnification of 10× and 20×, the numerical aperture of the objective being 0.5. The fluorescence signal is collected with a camera Coolsnap ES (Photometrix), specifically using the fluorescence excitation and emission filters. The images are assembled and processed using the Adobe Photoshop® software. With respect to the step of quantification, images of areas of each coverslip taken at random are recorded. Cells labelled with the antibodies anti-E MAb A4, anti-BVDV NS3 or anti-YFV E, are counted and recorded as infected cells. The total number of cells was counted by nuclear staining with DAPI. Infections are defined as the ratios of infected cells over the total number of cells.

### Testing for Cell to Cell Transmission of HCVcc Viruses.

The Huh-7 cells cultured in 24 well plates are infected with HCVcc over a period of 2 hours. The inoculum was eliminated and replaced by DMEM medium supplemented with GLUTAMAX®-1,10% of FCS and 1% low melting point agarose gel (SeaPlaque® Agarose marketed by LONZA®) containing EGCG (50 μM) or an equivalent volume of Dimethyl Sulfoxide (DMSO) as a control. The cells are incubated at 37° C. over a period of 3 days at the end of which the agarose is eliminated and the infection sites are detected using indirect immunofluorescence for HCV E1 protein as previously described.

### Experimentation Pertaining to Binding.

The Huh-7 cells were infected with JFH-Luc virus for 1 hour at 4° C. (attachment/binding step) in the presence of DMSO or 50 μM EGCG. The cells were washed with PBS and then incubated again for 1 hour at 40° C. (post-attachment/step of binding to the host cell receptors) in the presence of DMSO or 50 μM of EGCG. The cells were then washed to remove EGCG or DMSO and incubated for 1 hour at 37° C. in the presence of DMSO or 50 μM of EGCG (endocytosis/step of fusion). Finally, the cells were washed and cultured with DMEM medium (Invitrogen®) supplemented with L-alanyl-L-glutamine (GLUTAMAX-I™) and with 10% foetal calf serum at 37° C. for 48 hours. The level of infection was calculated by measuring the luciferase activity in cell lysates using the Renilla Luciferase Assay System kit marketed by PROMEGA®.

### Quantitative “Bindinq” Test.

HCVcc. For the quantitative “binding” experiments, the HCVcc virus was mass produced and purified by concentration and separation in a gradient of iodixanol. Supernatants of infected cells were precipitated with 8% PEG 6000 at 4° C. overnight and centrifuged for 25 minutes at 10 000 rpm. The pellets were resuspended in 1 mL, of PBS, loaded on a continuous iodixanol gradient (10-40%), and centrifuged at 36 000 rpm for 16 hours at 4° C. Fractions of 500 μL were collected and the titre of each fraction was determined. The most infectious fractions were combined in order to perform the experiments.

Quantitative “Bindinq” Test. The HCVcc virus was contacted with the Huh-7 cells for 1 hour at 4° C. in the presence of EGCG (50 μM), delphinidin chloride (50 μM) or porcine intestinal heparin (500 ug/mL). After 3 washes with PBS, the total RNAs (viral and cellular) were extracted using the NucleoSpin RNA II kit (Macherey-Nagel, Düren, Germany) and the HCV RNA quantified by quantitative RT-PCR (Castelain et al, J Clin Virol 2004). Heparin was used in parallel as a control.

### Example 1

### Test of the Effect of Different Green Tea Catechins on the HCV Virus Infection

Huh-7 cells were infected with the JFH1-Rluc virus for 2 hours in the presence of DMSO or 50 μM of different catechins namely: (+)-catechin (C), (−)-epicatechin (EC), (−)-epicatechin-3-gallate (ECG), (−)-epigallocatechin (EGC) and (−)-epigallocatechin-3-gallate marketed by EXTRASYNTHESE® (EGCG EXTRASYNTHESE®) or by CALBIOCHEM® (EGCG CALBIOCHEM®). These compositions of EGCG differ in their degree of purity EXTRASYNTHESE®>99%, CALBIOCHEM®>95%. DMSO is used as a solvent for the various catechins, it serves as a control test.

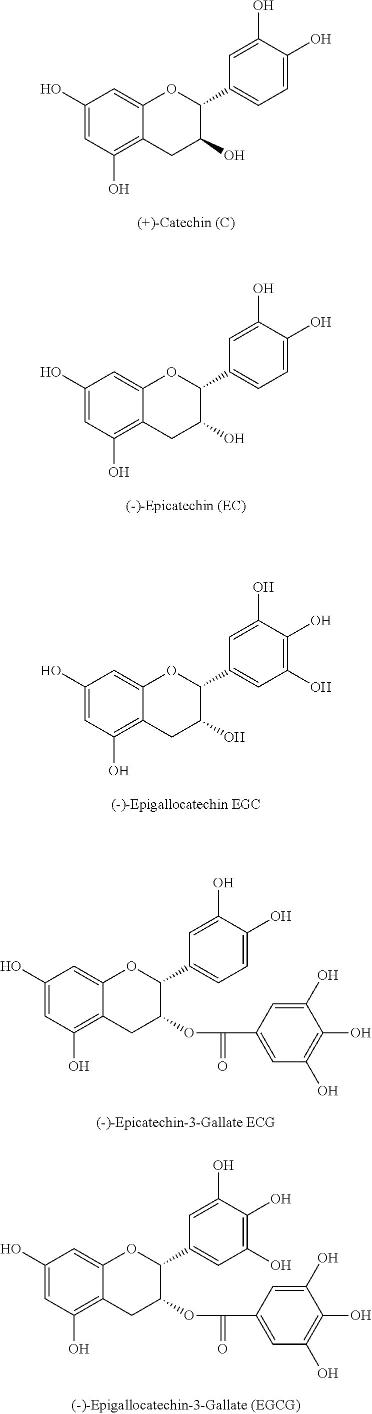 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1 ; O=C(O[C@@H]1Cc2c(O)cc(O)cc2O[C@@H]1c1cc(O)c(O)c(O)c1)c1cc(O)c(O)c(O)c1]

The cells were lysed 48 hours after the infection and expression of luciferase was quantified.

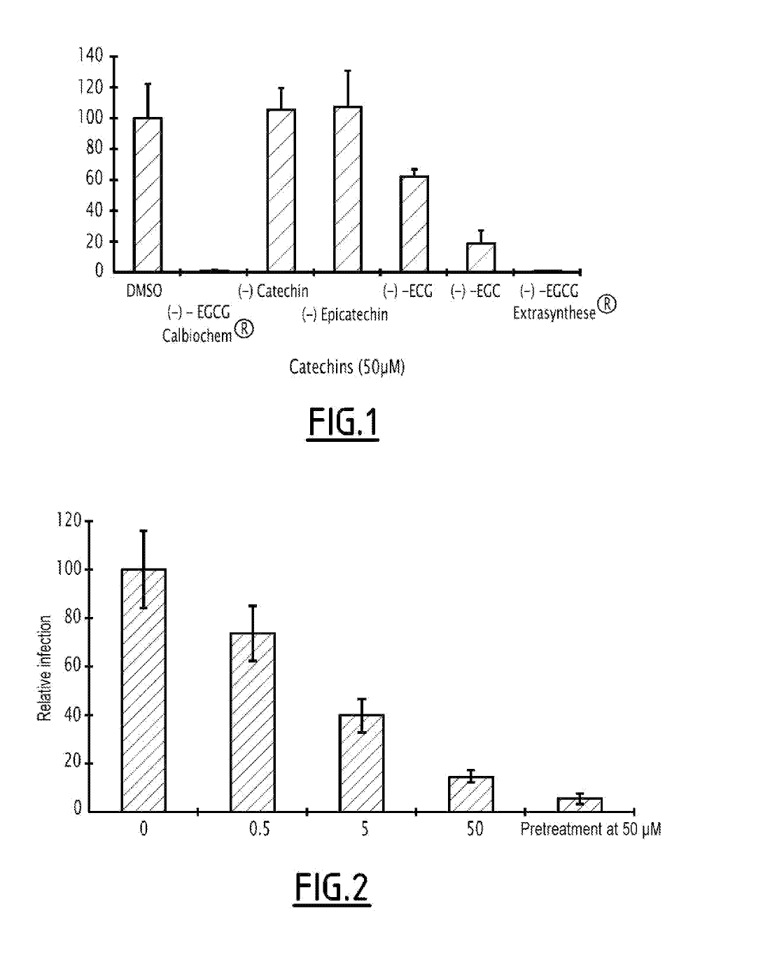

The measurement of the relative viral infection of Huh7 cells with the JFH1-Rluc virus based on the catechin present in the medium during the infection (FIG. 1), allows for clearly demonstrating that neither the (+)-catechin, nor the (−)-epicatechin induces inhibition of viral infection by the HCV virus. Indeed, the measurement of relative viral infection is identical to that observed for DMSO.

However, it should be noted that the EGCG be it that marketed by CALBIOCHEM® or EXTRASYNTHESE® shows a significant antiviral effect with inhibition of viral infection greater than 95%. With regard to the ECG and EGC molecules an antiviral effect is also found, however, with a lower rate of inhibition of viral infection on the order of about 40% and 80% respectively.

The measurement of the relative viral infection in the presence of ECG or EGC compared to EGCG, suggests an additive effect of the C2-3,4,5-trihydroxy-phenyl group of the chroman heterocycle and this same structure found in the C3 galloyl group of the chroman heterocycle, on the antiviral role of EGCG.

Given that the EGCG molecules whether they be those supplied by CALBIOCHEM® or EXTRASYNTHESE®, have an identical effect with respect to the inhibition of HCV infection, only the EGCG supplied by CALBIOCHEM® will be exemplified in the following sections.

### Example 2

Huh-7 cells cultured in vitro were infected for 2 hours with the JFH1—RLuc virus in the presence of increasing doses of EGCG (0; 0.5; 5; 50 μM) or with the virus previously incubated for 45 minutes with EGCG (50 μM). After infection, the cells were washed with DMEM culture medium supplemented with 10% foetal calf serum and incubated in the same medium. Forty-eight hours after infection, the cells were lysed and the Renilla luciferase activity was quantified by luminometer after incubation of 20 μL of cell lysate with the luciferase substrate.

FIG. 2 shows that the inhibition of viral infection by EGCG is dose-dependent.

At 50 μM EGCG decreases the viral infection of cultured Huh-7 cells by a factor greater than 10 (IC50=5 μM). However, viral replication is not affected by EGCG (results not shown). Therefore, EGCG inhibits the early stages of viral infection, that is, the steps enabling the entry of the virus into the cell.

In contrast, no effect of EGCG is observed on viral entry, if the non infected cells are treated with the molecule before or after infection. Therefore, the inhibition induced by EGCG acts directly on the virus and not on the host cell. This hypothesis is supported by the observation of an increase in the inhibitory effect of EGCG when the virus is incubated with the molecule before infection (pretreatment 50 μM).

### Example 3

The viral envelope proteins play a very key role in viral entry. Indeed, in addition to allowing interaction with the host cell receptor(s), these proteins make it possible to induce fusion between viral and cellular envelopes. In order to study the effect of EGCG on the initial stages of infection by the HCV virus, a reference study model was used allowing for the expression of the viral envelope glycoproteins of interest: these are the infectious virus like particles.

Different genotypes of the HCV virus are present in the global population. Thus, in order to determine the specificity of the inhibitory effect of EGCG with respect to the viral genotype of HCV, different genotypes of envelope glycoproteins E1 and E2 of the HCV virus were studied. Production was undertaken of infectious virus like particles (HCVpp) expressing on their surface the envelope proteins E1 and E2 of the HCV virus of genotypes 1a, 1b, 2a, 2b, 3, 4, 5, and 6; and, by way of a control, the virus like particles of the VSV virus expressing the envelope protein VSV (VSVpp). The various virus like particles produced express luciferase so as to quantify the infection.

In order to do this Huh-7 cells were infected for a period of 2 hours with the HCVpp virus like particles of the various different genotypes tested or with VSVpp virus like particles in the presence of EGCG at 50 μM or DMSO. DMSO being the solvent of EGCG, it is used as a control. At forty-eight hours post-infection, the cells were lysed and the luciferase activity was quantified by luminometer after incubation of 20 μL of cell lysate with the luciferase substrate.

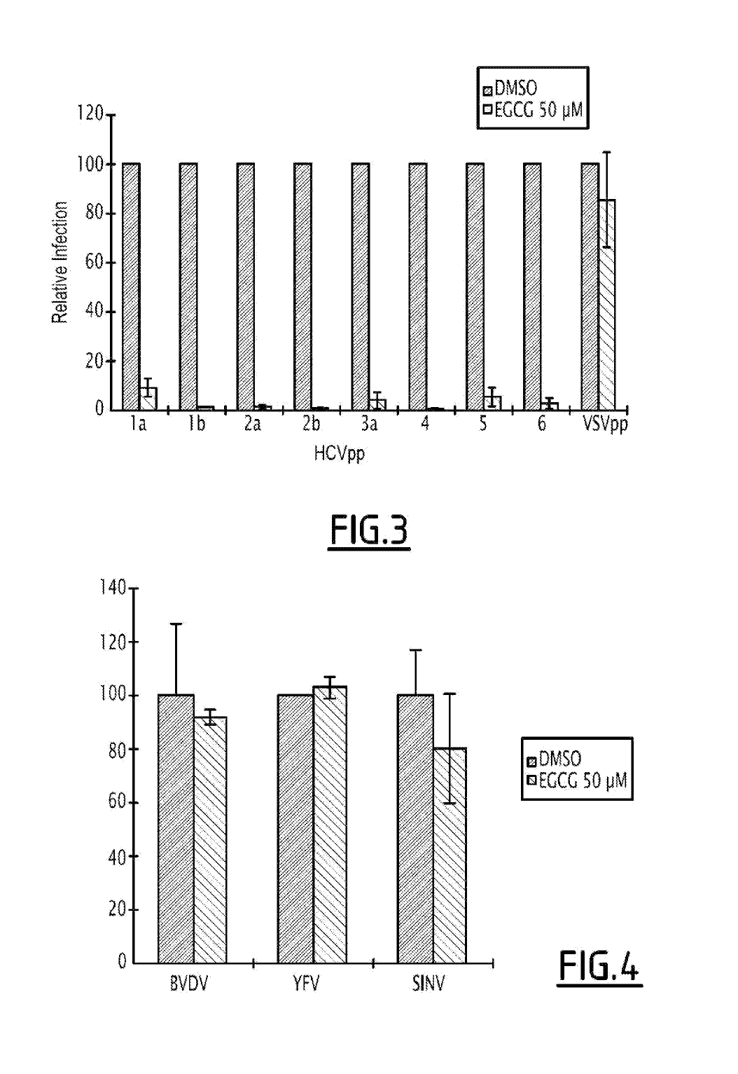

Although having no effect on infection by the VSVpp, EGCG (at 50 μM) has an inhibitory effect on the infection by HCVpp of all genotypes tested (FIG. 3).

Accordingly, the inhibitory effect of EGCG directly involves the viral envelope glycoproteins. Thus, inhibition of viral entry mediated by EGCG is either due to the inhibition of the step of attachment, that is the initial interactions with the host cell receptor(s), or inhibition of the step of fusion between the viral and cellular envelopes, or both these two steps.

Furthermore, the use of HCVpp expressing the viral glycoproteins of different viral genotypes shows that EGCG is an effective antiviral agent of HCV regardless of the genotype tested. It may be observed that there is substantially nil relative infection in the presence of EGCG, upon infection by HCVpp of genotype 1b, 2a, 2b and 4. The relative infections observed were very low for the genotypes 1a, 3a, 5 and 6, that is between 5% and 13%. Thus, although an infection by VSVpp induces the measure of a relative infection rate of 85% in the presence of EGCG, an infection by HCVpp whichever be the genotype concerned induces the measure of a relative infection rate of between 13% to 0% in the presence of EGCG. These results show the strong potential of EGCG as an antiviral in the treatment of hepatitis C whichever be the viral genotype involved. In addition, it is important to note that the HCV genotype 1 which is the most resistant to current treatments and the majority genotype in the European and American population is also inhibited by EGCG.

### Example 4

Example 2 demonstrates the antiviral nature of EGCG with respect HCV regardless of its genotype. Example 2 demonstrates in addition, that this effect is specific to HCV since EGCG does not inhibit VSV infection. However, while HCV is a virus with a single stranded RNA genome of positive polarity, VSV is a non segmented negative sense RNA virus.

Thus, in order to determine the level of specificity of EGCG within even single stranded positive RNA viruses, infection experiments in the presence of EGCG were carried out with other viruses of the family Flaviviridae.

Testing was performed on the yellow fever virus (YFV), the Sindbis virus (SINV) and the bovine viral diarrhoea virus (BVDV). Infections were carried out either in the presence of DMSO or EGCG at 50 μM. Cells infected with these various viruses were the Huh-7 for YFV or SINV and MDBK cells for the BVDV. The quantification of the infection was carried out by immunofluorescence after labelling of the infected cells with an anti-E (2D12) antibody for the YFV virus or anti-NS3 (osc23) antibody for the BVDV virus. A step of detection with a Cy3 labelled secondary antibody is then performed. Regarding the SINV virus, with the latter expressing luciferase, the measurement of the relative infection was performed by quantification of luciferase.

Quantification of infection based on the presence of EGCG or DMSO in the medium during infection of cells with the YFV, BVDV and SINV viruses (FIG. 4) clearly shows that EGCG does not have an inhibitory effect on cell infection by any of these viruses.

Consequently, EGCG is therefore not an antiviral for the other members of the family Flaviviridae. The antiviral effect of EGCG is specific to HCV.

### Example 5

The results described here above show that EGCG inhibits viral entry into cultured cells. A second mode of viral infection is the transmission of the virus from cell to cell. In order to determine whether EGCG also inhibits the spread of the virus from cell to cell, Huh-7 cells were infected with JFH1 virus for a period of 2 hours, and then incubated in medium containing 1% agarose in the presence or absence of EGCG (at 50 μM). The agarose prevents infection of cells by the virus secreted into the medium and thus makes it possible to observe the cell to cell transmission of the virus by virtue of the cells being in contact amongst themselves.

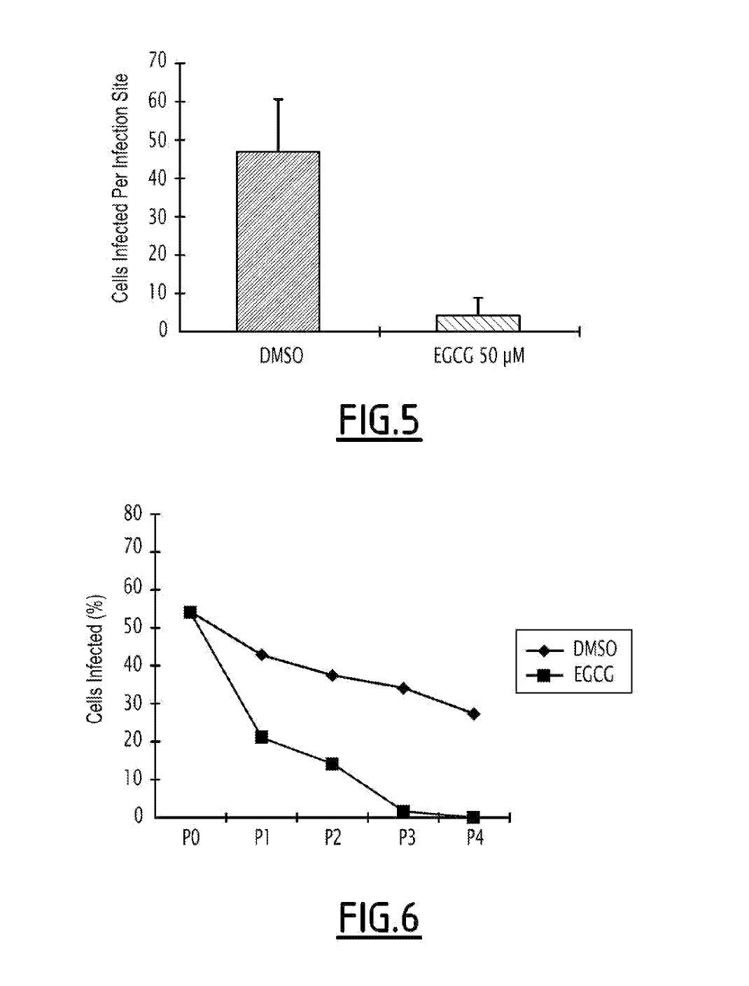

At seventy-two hours post-infection, the cells were fixed and infection was detected by immunofluorescence after labelling with an anti-E1 antibody. A step of detection is then carried out with a Cy3 coupled secondary antibody. The nuclei are stained with DAPI staining. The number of cells per infection site was quantified (FIG. 5). In the presence of EGCG the infection sites are much smaller. They contain an average of 3-4 infected cells showing that the virus has not propagated after infecting a cell. In contrast, the control sites contain an average of 45 infected cells. In view of the foregoing, EGCG is an inhibitor of the cell to cell propagation of the HCV virus.

Thus EGCG appears to be a good candidate as an antiviral to prevent reinfection of a healthy liver after transplantation thereof in an infected patient.

### Example 6

Experiments involving successive infection in the presence or absence of EGCG from infected cell supernatants were conducted so as to test the efficacy of EGCG on the virulence of a supernatant of infected cells, as well as its capacity for eradication of the virus from infected culture supernatants. Huh-7 cells were infected with the virus HCV JFH1 (P0). DMSO or 50 μM of EGCG was added post-infection. The culture supernatant was collected after 48 hours and then used to infect healthy cells in the presence of DMSO or 50 μM of EGCG (P1). The supernatant of this culture (P1) was in turn collected after 48 hours and used to re-infect healthy cells (P2). This procedure is reproduced two more times (up to P4). The number of cells infected was quantified in each passage by immunofluorescence after labelling the cells with an antibody directed against the viral envelope protein E1 (A4) antibody. The total number of cells is quantified by counting the nuclei after staining by DAPI marking.

The study of the percentage of cells infected in each passage of a supernatant from one culture to another depending on the addition of EGCG or DMSO (FIG. 6), shows that EGCG allows elimination of HCV from the culture supernatant from the third passage onwards and the complete elimination in the fourth passage. This supports the conclusion that EGCG is a good candidate as a curative molecule in patients infected with HCV.

### Example 7

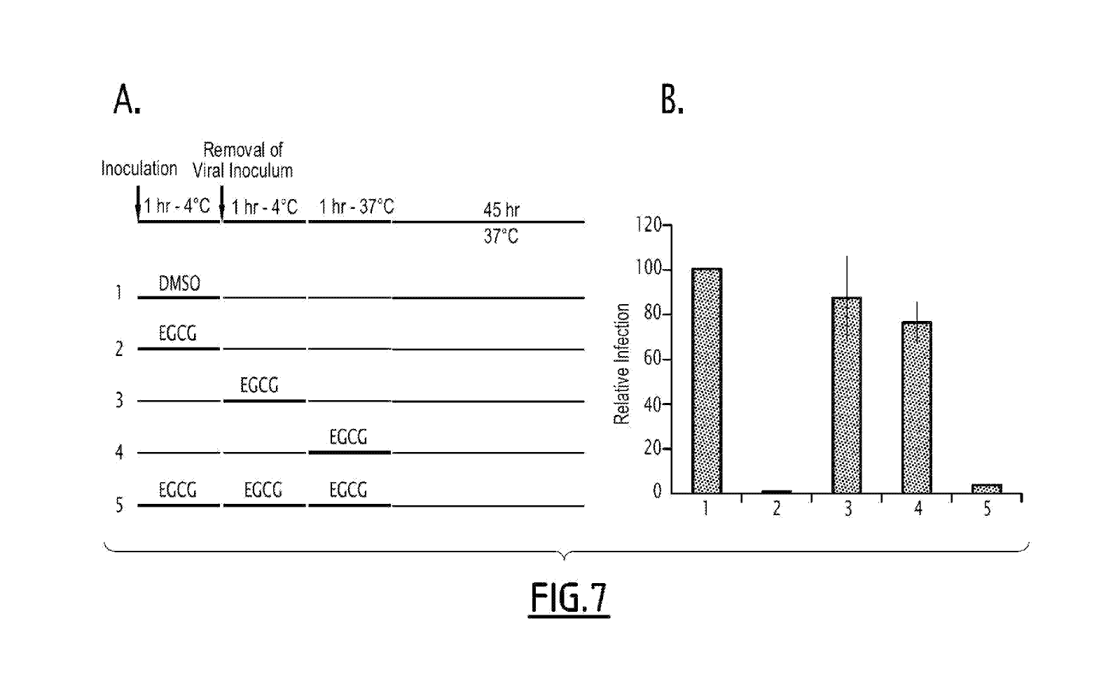

Entry of the HCV virus into the host cell is composed of three steps i) the attachment of the virus or infected host cell to the surface of the non infected host cell, ii) interaction with the specific viral receptor(s) and iii) fusion of the viral lipid envelope and the infected host cell (endosomal or plasma membrane) enabling the introduction of the viral genome in the cytoplasm of the infected cell. In order to determine which of these steps is inhibited by EGCG, the viral entry was split up in a manner so as to study separately the inhibitory effect of EGCG on each of these steps (see FIG. 7 A).

The step of attachment of the viral particles to cells is effected by putting the cells in contact with the virus followed by a one hour of incubation at 4° C. The second step is carried out by incubating the cells for one hour at 4° C. after removal of the viral inoculum, thereby allowing the virus attached to strengthen their bond with their receptors. At 4° C., the process of endocytosis is blocked. Thus, the third step of endocytosis and fusion of viral and cellular membranes is carried out at 37° C. for one hour.

DMSO or 50 μM of EGCG are added during the different steps according to the relevant samples as shown in the diagram (FIG. 7A). Thus, Sample 1 is the control sample which received DMSO during the first step. The other samples correspond to the addition of 50 μM of EGCG during the step of attachment (Sample 2); interaction with receptors on the host cell (Sample 3); endocytosis and fusion of the viral lipid envelope and the infected host cell (Sample 4); or during all three steps of the viral entry (Sample 5).

The cells were thereafter incubated for 45 hours at 37° C. and then lysed so as to quantify the luciferase activity. Infection is expressed as a percentage of the luciferase activity measured without EGCG. The experiments were performed in triplicate and the values given correspond to the average values of these three different experiments.

As shown in FIG. 7B, a significant reduction of infection is observed when EGCG is added from the step of attachment (Samples 2 and 5). On the other hand. EGCG does not seem to have any effect on the step of interaction with the host cell receptors (Sample 3) or during the step of endocytosis or fusion (Sample 4). Taken together, all of these results demonstrate that EGCG inhibits the early steps of HCV binding to the plasma membrane of the host cell, that is to say, the step of attachment of the HCV virus to the host cell.

### Example 8

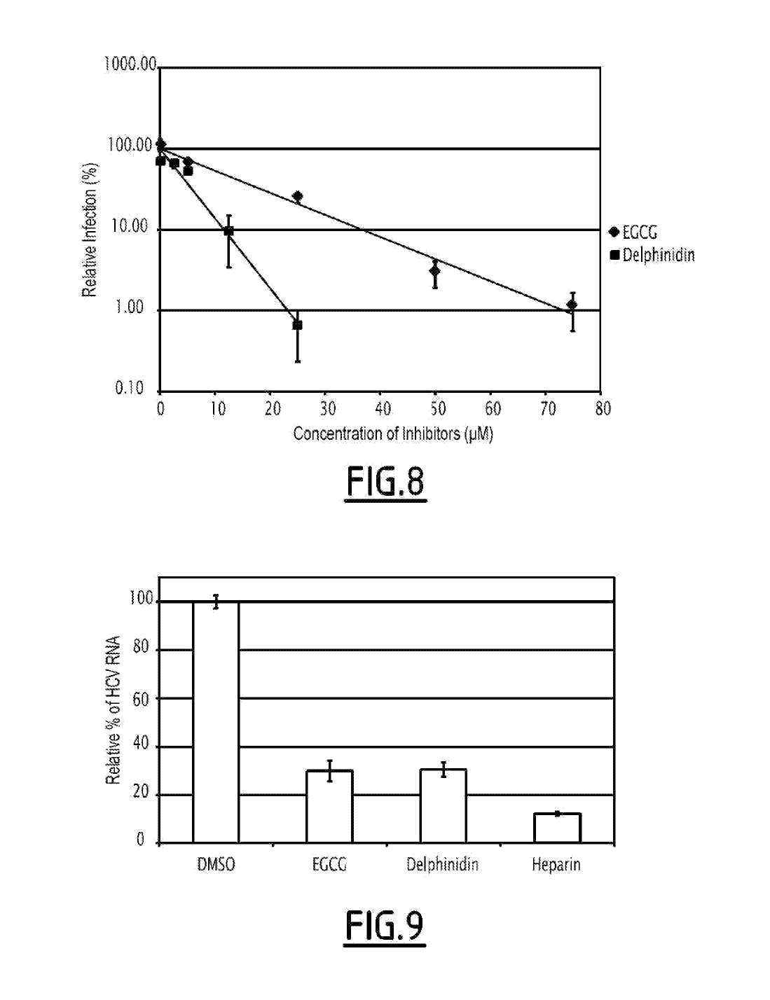

The Huh-7 cells were infected with HCVcc for a period of 2 hours in the presence of increasing doses of EGCG or delphinidin chloride. The rate of infection was determined 34 hours after inoculation by detecting the envelope protein E1 by immunofluorescence using an anti-E1 antibody (A4) and then an anti-mouse IgG secondary antibody conjugated to the fluorochrome Cy3. This experiment shows that delphinidin chloride, like EGCG, inhibits the infection of cells with HCVcc in a dose dependent manner (FIG. 8). The half maximal inhibitory concentration (concentration inhibiting 50% infection—IC50) was determined in these experiments. The IC50 of EGCG was calculated to be about 11 μM whereas the IC50 of delphinidin chloride is about 3.5 μM.

These results show on the one hand, that delphinidin chloride is a new inhibitor of HCV entry into cells and on the other hand, that this molecule has a higher efficacy as compared to EGCG as an anti-HCV antiviral agent.

### Example 9

Example 3 demonstrates that EGCG inhibits an early step of HCV entry into cells mediated by the surface glycoproteins of HCV.

In order to determine whether the step of attachment to the cell surface was inhibited by EGCG, quantitative “binding” (or attachment) tests have been carried out. The action of delphinidin chloride was tested in parallel. Heparin, a known inhibitor of HCV attachment to the cell surface, was used as a positive control. The cells were incubated with HCVcc purified to a multiplicity of infection of 10 for 1 hour at 4° C. in the presence of DMSO, 50 M of EGCG, 50 μM of delphinidin chloride or 500 μg/mL of heparin. The cells were washed 3 times with cold PBS and the total RNA extracted. The amount of virus fixed was determined by quantifying the viral genomic RNA by means of quantitative RT-PCR. As expected, the attachment of the virus to the surface of cells in the presence of heparin is greatly reduced (FIG. 9). In a similar manner, in the presence of EGCG or delphinidin chloride, a strong decrease in virus attachment to the cell surface is also observed. All of these results show that both EGCG and delphinidin chloride probably by acting directly on the virus particle, inhibit viral entry by preventing the attachment of HCVcc at the cell surface.

What is claimed is:

1-17. (canceled)

18. An ex vivo method for reduction of the infectivity or inactivation of a hepatitis C virus (HCV), said method comprising a step of contacting said hepatitis C virus with a compound having the formula (I): 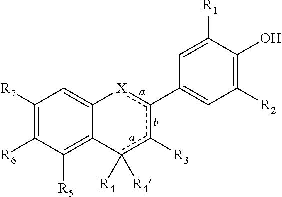
wherein:
X is O when a is a single bond or O⁺ when a is a double bond,
R1 and R2 are independently of each other a hydrogen atom, a hydroxyl group or a methoxyl group,
R3, R5 and R7 are independently of each other, a hydrogen atom, an OH group, an O-glycosyl group, a (C1-C18) alkoxyl or a group having the formula (II): 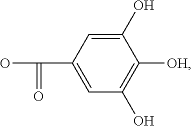 [SMILES: O=C(O)c1cc(O)c(O)c(O)c1]
R4 and R6 are independently of each other, a hydrogen atom or an OH group,
R4′ is a hydrogen atom or R4′ is with R4 and the carbon atom to which they are bound, a C═O group, when a is a single bond or R4′ is nothing when a is a double bond,
a and b are identical or different, being either a single bond or a double bond,
provided that at least one of the R3, R5 and R7 groups is a group having the formula (II) and/or R1 and R2 are both OH groups,
or one of the pharmaceutically acceptable salts or esters thereof, said compound having the formula (I) being in the form of a pure stereoisomer or in the form of a mixture of enantiomers and/or diastereomers, including racemic mixtures.

19. An ex vivo method of inactivation according to claim 18, wherein the said HCV is present in a biological sample.

20. A method of treatment or prevention of HCV infection, comprising the administration to an individual in need thereof of a therapeutically effective amount of a compound having the formula (I) 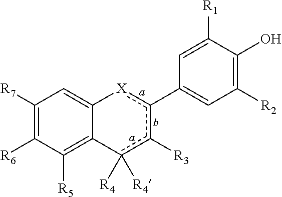
wherein:
X is O when a is a single bond or O⁺ when a is a double bond,
R1 and R2 are independently of each other a hydrogen atom, a hydroxyl group or a methoxyl group.
R3, R5 and R7 are independently of each other, a hydrogen atom, an OH group, an O-glycosyl group, a (C1-C18) alkoxyl or a group having the formula (II): 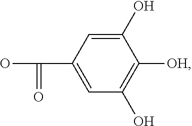 [SMILES: O=C(O)c1cc(O)c(O)c(O)c1]
R4 and R6 are independently of each other, a hydrogen atom or an OH group,
R4′ is a hydrogen atom or R4′ is with R4 and the carbon atom to which they are bound, a C═O group, when a is a single bond or R4′ is nothing when a is a double bond,
a and b are identical or different, being either a single bond or a double bond,
provided that at least one of the R3, R5 and R7 groups is a group having the formula (II) and/or R1 and R2 are both OH groups,
or one of the pharmaceutically acceptable salts or esters thereof, said compound having the formula (I) being in the form of a pure stereoisomer or in the form of a mixture of enantiomers and/or diastereomers, including racemic mixtures.

21. The method of claim 20, wherein said compound is an antiviral agent for inhibiting one or both of the infection of a host cell with HCV and the transmission of HCV from an infected host cell to another host cell.

22. The method of claim 21, wherein said compound is an antiviral agent for inhibiting the entry of hepatitis C virus into a host cell.

23. The method of claim 20 wherein said compound is an antiviral agent for inhibiting the adhesion of the hepatitis C virus, or a host cell infected with the hepatitis C virus, to the membrane of a non infected host cell.

24. The method of claim 21, wherein said host cell is a hepatocyte cell.

25. The method of claim 20, wherein said compound is an antiviral agent for inhibiting the interaction of the surface glycoproteins of HCV with at least one target protein in a host cell.

26. The method of claim 25, wherein said compound is an antiviral agent for inhibiting the interaction of one or both of the glycoproteins E1 and E2 of HCV with at least one target protein of a host cell.

27. The method of claim 20 wherein said individual is resistant or intolerant to treatment for an HCV infection, by one or both of an immunomodulatory agent and an inhibitor of viral metabolism.

28. The method of claim 20, wherein said compound has the formula (III): 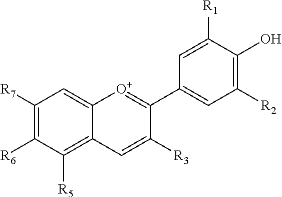
wherein:
R1 and R2 are independently of each other a hydrogen atom, a hydroxyl group or a methoxyl group,
R3, R5 and R7 are independently of each other, a hydrogen atom, an OH group, an O-glycosyl group, a (C1-C18) alkoxyl or a group having the formula (II) 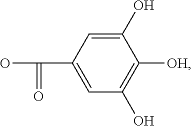 [SMILES: O=C(O)c1cc(O)c(O)c(O)c1]
R6 is a hydrogen atom or an OH group, and
at least one of the R3, R5 or R7 groups is a group having the formula (II), or R1 and R2 are both OH groups,
or one of the pharmaceutically acceptable salts or esters thereof.

29. The method of claim 20 wherein said compound has the formula (IV) 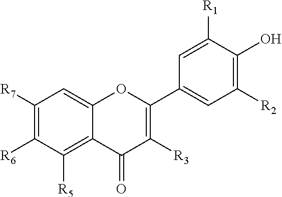
wherein:
R1 and R2 are independently of each other a hydrogen atom, a hydroxyl group or a methoxyl group,
R3 is an OH group, an O-glycosyl group, a (C1-C18) alkoxyl or a group having the formula (II) 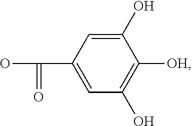 [SMILES: O=C(O)c1cc(O)c(O)c(O)c1]
R5 and R7 are independently of each other, a hydrogen atom, an OH group, an O-glycosyl group, a (C1-C18) alkoxyl or a group having the formula (II),
R6 is a hydrogen atom or an OH group, and
at least one of the R3, R5 or R7 groups is a group having the formula (II), or R1 and R2 are both OH groups,
or one of the pharmaceutically acceptable salts or esters thereof.

30. The method of claim 20 wherein said compound has the formula (V) 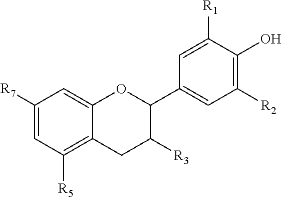
wherein:
R1 and R2 are independently of each other a hydrogen atom, a hydroxyl group or a methoxyl group,
R3 is an OH group, an O-glycosyl group, a (C1-C18) alkoxyl or a group having the formula (II) 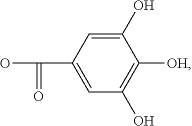 [SMILES: O=C(O)c1cc(O)c(O)c(O)c1]
R5 and R7 are independently of each other, a hydrogen atom, an OH group, an O-glycosyl group, a (C1-C18) alkoxyl or a group having the formula (II), and
at least one of the R3, R5 or R7 groups is a group having the formula (II), or R1 and R2 are both OH groups,
or one of the pharmaceutically acceptable salts or esters thereof.

31. The method of claim 20 wherein said compound has the formula (I) 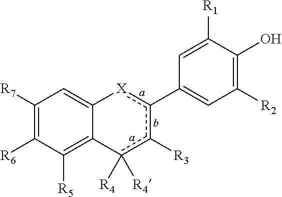
wherein,
X is O when a is a single bond or O⁺ when a is a double bond, a and b are identical or different, being either a single bond or a double bond,
the R1, R5 and R7 groups are hydroxyl groups,
R2 is a hydroxyl group or a hydrogen atom,
R3 is a hydroxyl group, a hydrogen atom or a galloyl group having the formula (II) 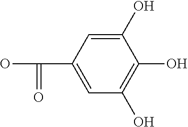 [SMILES: O=C(O)c1cc(O)c(O)c(O)c1]
R4′ is hydrogen atom when a is a single bond or R4′ is nothing when a is a double bond, and
R6 is a hydrogen atom,
or one of the pharmaceutically acceptable salts or esters thereof.

32. The method of claim 20 wherein said compound is selected from the group consisting of anthocyanidins, anthocyanins, flavonols, and flavan-3-ols.

33. The method of claim 20 wherein said compound is an anthocyanidin selected from the group consisting of Delphinidin, Pulchellidin or Tricetinidin, or a Flavan-3-ol selected from the group consisting of Epigallocatechin (EGC), Epigallocatechin Gallate (EGCG), Gallocatechin (GC), Catechin Gallate (CG), Gallocatechin Gallate (GCG), Epicatechin (EC), Epicatechin Gallate (ECG), the pharmaceutically acceptable salts and esters thereof, and mixtures thereof.

34. The method of claim 20 wherein said compound is Epigallocatechin (EGC), Epicatechin Gallate (ECG), Epigallocatechin Gallate (EGCG), Delphinidin or one of the pharmaceutically acceptable salts or esters thereof.

35. The method of claim 20 wherein said compound is co-administered with an additional agent to said individual.

36. The method of claim 20, wherein said compound is the only antiviral agent administered to said individual.
